SEQUENCE LISTING (2 sequences)

SEQ ID NO: 1 - DNA, 50 bp, Mus musculus
cttccctcatcctccagcaggctagctcgggcttggtcacctgacagggg

SEQ ID NO: 2 - DNA, 44 bp, Mus musculus
caagaaggggactcggcttgtctgaactagcataaggaagtccc

(19) United States
(12) Patent Application Publication
(10) Pub. No.: US 2006/0292560 A1
(43) Pub. Date: Dec. 28, 2006

(54) Transcription factor target gene discovery

(76) Inventor: Robert Burgess, Temecula, CA (US)

Correspondence Address: TransGenetics, Inc., 905 Sagebrush Trail, Garland, TX, 75044, US

(21) Appl. No.: 10/275,846

(22) PCT Filed: Aug. 8, 2001
(86) PCT No.: PCT/US01/24823

§ 371 Date: Apr. 29, 2005

Related U.S. Application Data
(60) Provisional application No. 60/225,225, filed on Aug. 14, 2000.

(51) Int. Cl. C12Q 1/68; G06F 19/00; C12P 19/34; C12N 9/22; C07K 14/705

(52) U.S. Cl. 435/6; 530/352; 702/20; 435/91.2; 435/199

(57) ABSTRACT

The ability to rapidly define transcription factor target genes allows for the study of genetic cascades involved in development, physiology and disease. The presently descibed invention outlines the combination of novel chromosomal immunoprecipitation and molecular biology technologies fot the high-throughput in vico discovery and characterization of both known and unknown transcription factor target genes. Through an application of solid phase support matrices and sequential chromosomal immunoprecipitation in combination with molecular cloning procedures this methodology allows for the rapid, stringent purification from cells and tissues of nucleotide sequences representing targets for regulation by transcription factors. Implementation of the technology described herein will result in the efficient simultaneous characterization of both regulatory element and coding sequence target gene information and will be valuable for assessing genetic hierarchies and developing therapeutics.

### 1.0 FIELD OF THE INVENTION

The following invention describes the utilization of solid matrix binding technology in combination with sequential chromosomal immunoprecipitation and molecular cloning technologies to discover and characterize transcription factor target genes.

### 2.0 BACKGROUND OF THE INVENTION

The specific regulation of transcription within the nucleus of the cell is one of the basic facets of the cellular machinery and is known to be the implicit foundation behind all cellular characteristics. The ability to differentially regulate the activity of each of the estimated 26,000 genes depends upon the presence or absence of various transcriptional activator and/or repressor proteins (Venter et al., Science, 2001, 291(5507): 1304-1351). FIG. 1 illustrates this concept for the steroid receptor class of transcription factors. Steroid receptors, represented by the rectangles, dimerize and bind to target gene regulatory regions. In the presence of a steroid ligand depicted by the ovals, target genes are activated. In the absence of ligand the receptor is bound to corepressor machinery and target genes are inactive.

Interactions between these as well as other factors and their target loci have evolved over time into a complex series of temporal and biochemical events which governs transcription under tight regulatory constraints (for review see Semenza et al., Human Mutations, 1994, 3:180-199). It is the interaction between these factors and sequence-specific regulatory elements that has shed insight into the mechanisms by which cells keep such entities as cell division, differentiation and immunomodulation in check By deciphering these genetic cascades and ultimately defining transcription factor targets it will be possible, for example, to determine just how tumor suppressing transcription factors such as p53 (Zambetti et al., Genes Dev., 6: 1143-1152, Zauberman et al., Oncogene, Jun. 15, 1995;10(12): 2361-6) and Rb (friend et al., Proc. Natl. Acad. Sci. USA, 1987, 84: 9059-9063) exert their effects on both inhibiting cell division and promoting cell death. Indeed, the value of transcription factor/regulatory interactions is evidenced by the wealth of patents recently issued in the United States relating directly to the factors themselves or technology pertaining to gene regulation (a representative set of examples includes U.S. Pat. Nos. 5,53,036, 5,858,973, 5,863,757, 5,880,261 and 6,117,638 herein incorporated by reference).

The identification of transcription factor target loci requires an assessment of protein/DNA interactions in vivo. The chromosomal immunoprecipitation (ChIP) assay has been demonstrated as a method which successfully allows for the purification of in vivo protein/protein interactions which occur in combination with DNA regulatory elements as well as direct protein/DNA interactions from cellular extracts of either cytoplasmic or nuclear origin (Solomon et al., Cell, 1988, 53: 937-947; de Belle et al., Biotechniques, 2000, 29(1): 170-175). It is based upon the chemically catalyzed cross-linkage of biochemical interactions in living cells followed by purification of desired complexes from nonspecific contaminants. To date, use of the ChIP assay has proven to be of value for the assessment of transcription factor complex recruitment to particular nucleotide sequences of known origin. By determining the presence or absence of a particular transcription factor on a known DNA sequence or binding site present within a particular gene, for example, it is possible to establish whether specific known genes are targets for regulation by chosen factor. However, in order to identify previously uncharacterized or undiscovered targets for potential regulation by a particular transcription factor a number of advances in the technology must be achieved. For example, efficient recovery of quantities of DNA large enough to allow for cloning and sequencing of the potential transcription factor targets must occur. In addition, an optimization for the opportunity to isolate transcribed portions of genes and eliminate noncoding genomic sequences which often do not reveal the identity of the target gene must be accomplished. Finally, high-throughput organization of sequences obtained into a searchable database format should be undertaken to provide for maximum utility of the discovery transcription factor target genes. Incorporation of the modified, significantly improved ChIP assay into the described present invention in combination with molecular cloning methods now allows for the high-throughput isolation and characterization of both known and unknown transcription factor target loci. In addition, these modifications and improvements increase the sensitivity of target gene retrieval while simultaneously reducing background.

Solid phase technology has had a significant impact on the efficiency and sensitivity of protein complex purification. Compounds such as sepharose and magnetic beads have allowed for extensive purification and characterization of protein/protein complexes from both in vitro and in vivo mixtures, without compromising the quantitative or qualitative aspects of samples obtained Dynal Corporation Technical Handbook, 1998, Sigma Corporation, cat. #4B-200). It's application for the purposes of identifying transcription factor target genes of unknown origin and in a high-throughput format, however, has yet to be implemented. It is the use of solid phase technology in the presently described invention which significantly increases the sensitivity of obtaining real in vivo targets for transcription factors while reducing background false positive sequences obtained.

Through both a combination and modification of the above technologies as well as other molecular biology techniques such as exon scanning, inverse PCR and cDNA library screening the presently described invention allows for the extensive and exhaustive characterization of transcription factor target genes of both known and unknown origin and of a direct (the gene is bound by the factor) and indirect (interaction through other proteins) nature. It is the implementation of chromosomal immunoprecipitation procedures improved via the use of-solid phase support and sequential immunoprecipitation for multiple proteins which permits the potential complete and thorough analysis of a great deal of the transcriptional cascades present in the nucleus of the cell. The proposed technology described herein is applicable to a very limited quantity of cell or tissue samples, which makes it suitable for clinical analysis and comprehensive medical diagnostics. The utilization of this technology will no doubt have a significant impact on the fields of therapeutics, medical diagnostics and basic research related to the realm of transcriptional regulation.

### 3.0 SUMMARY OF THE INVENTION

The use of chromosomal immunoprecipitation (ChIP) for the identification of targets for transcriptional regulation by transcription factors has been limited by both the insensitivity of the technology to eliminate considerable nonspecific protein/DNA interactions as well as to the discovery and characterization of only previously identified nucleotide sequences. The presently described invention overcomes the above limitations of chromosomal immunoprecipitation by employing a combination of novel sequential immunoprecipitation procedures utilizing antibodies to the basal transcriptional machinery, solid phase separation procedures and extensive cloning applications including a modified and significantly improved version of inverse PCR which allow for the discovery of target genes and their regulatory elements. The combination of improved chromosomal immunoprecipitation procedures with expression profiling and cloning technologies described in the present invention has allowed for the discovery and characterization of both known and unknown target sequences for chosen transcription factors. In addition, the presently described technology is highly automatable, allowing for extensive high-throughput analysis of virtually any genetic cascade.

One embodiment of the present invention is the formaldehyde fixation reaction process which cross-links DNA binding proteins with their prospective nucleotide binding sites present within close proximity or distal to target genes in living cells and or tissues. This fixation reaction is designed and customized specifically for each particular cell line and/or tissue being studied.

An additional embodiment of the present invention is other chemical methods utilized for the purposes of fixing and/or cross-linking proteins to their prospective target nucleotide sequences in vivo directly through interaction with DNA or indirectly utilizing protein-protein contacts.

Another embodiment of the present invention is the cross-linked protein/target gene complex created by the formaldehyde crosslinkage reaction in vivo. Said complex theoretically contains a mixture of protein/DNA complexes containing the desired transcription factor or regulatory protein directly or indirectly bound to its prospective target loci.

Another embodiment of the present invention is an antibody which is specific for Drosophila melanogaster or Sciara coprophila RNA Polymerase II protein large subunit. The antibody may be of monoclonal or polyclonal origin and may recognize similar epitopes from different species.

Yet another embodiment of the present invention is an antibody which binds specifically to the mammalian transcription factor p53. Said antibody may be of monoclonal or polyclonal origin.

Still another embodiment of the present invention is an antibody-linked to magnetic beads which binds specifically to either Drosophila melanogaster or Sciara coprophila RNA polymerase II protein large subunit. It is the solid-phase support linkage which enhances recovery and specificity of target chromatin upon immunoprecipitation.

Another embodiment of the present invention is an antibody which is linked to magnetic beads which binds specifically to the mammalian p53 protein.

Yet another embodiment of the present invention is the recovered fraction of the cross-linked, fixed chromatin protein/DNA complex.

Another embodiment of the present invention is the sonicated chemically cross-linked protein/DNA complex isolated after sonication but prior to immunoprecipitation. Sonication allows for efficient immunoprecipitation of DNA fragment sizes small enough to be characterized in a high-throughput format via polymerase chain reaction (PCR) or other molecular biology techniques.

Still another embodiment of the present invention is the immunoprecipitated protein/DNA complex prior to release of the antibody and reversal of cross-linkage isolated utilizing antibodies which recognize either the Drosophila melanogaster or Sciara coprophila RNA polymerase II large subunit as well as the mammalian p53 protein.

An additional embodiment of the present invention is the sequential immunoprecipitation of cross-linked protein/DNA complexes from living cells and tissues utilizing antibodies to core transcriptional machinery factors first and to specific transcription factors second. Sequential immunoprecipitation eliminates the majority of nontranscribed sequences and satellite DNA by focusing only upon transcribed and/or actively regulated genes. It is primary immunoprecipitation with antibodies to proteins found in the basal transcriptional apparatus which results in increased sensitivity through a reduction in the amount of nontranscibed genomic DNA pulled down during subsequent immunoprecipitation reactions. Theoretically only actively transcribed genetic sequences are present as templates for the second round of immunoprecipitation. Secondary immunoprecipitation with antibodies to specific transcription factors is thereby significantly more efficient as is described herein and allows for the opportunity to characterize transcription factor function with respect to the regulation of gene activity. These sequential rounds of immunoprecipitation may be performed in any order with the similar result of increased sensitivity for the discovery of transcription factor target genes and decreased background or nonspecific sequences obtained.

Additionally, solid phase chromosomal immunoprecipitation eliminates loss of cross-linked protein/DNA complex material initially precipitated from cellular extracts by providing a solid support and thereby enhances the potential ability to recover target DNA fragments and hence the nucleotide sequences corresponding to these fragments. Excessive loss is prevented through clean, efficient recovery of antibody/protein/DNA complexes due to tight linkages between the solid phase (beads in the case of the present invention) and antibodies.

Yet another embodiment of the present invention is the utilization of polymerase chain reaction (PCR) to detect known target loci within the collection of pull-down fragments which putatively contains both known and unknown target genes. It is the detection and monitoring of known controls which allows for a characterization of the efficiency of the system.

As well, an additional embodiment of the present invention is the utilization of inverse PCR (I-PCR) in combination with solid phase sequential chromosomal immunoprecipitation for purposes of defining only direct targets for regulation by specific transcription factors as well as for background reduction. Specifically, oligonucleotides corresponding to transcription factor binding sites are used to PCR flanking sequences present in DNA fragment populations isolated by the technology described herein. The application of this modified version of I-PCR to sequentially immunoprecipitated chromosomal templates hence results in the discovery and cloning of direct targets for regulation by the transcription factor in question.

Another embodiment of the present invention is the facilitated cloning of both known and unknown target genes from DNA fragments isolated by the presently described methods. These potential targets, for transcription factors of DNA binding and nonDNA binding origin, are cloned through successive rounds of screening against cDNA libraries and genomic DNA libraries, ligation and transfer into bacteriophage and/or plasmid vectors, polymerase chain reaction including but not limited to I-PCR and DNA sequencing.

Yet another embodiment contemplated by the present invention is the screening of immunoprecipitated DNA fragments potentially containing target loci against libraries, arrays and/or microarrays of both known and unknown genes. These libraries and arrays may be of either cDNA or oligonucleotide composition. It is the screening of immunoprecipitated DNA fragments against these libraries, arrays and microarrays which facilitates the discovery of target genes for the transcription factor being studied. Said screen allows for a rapid identification of coding sequences for transcription factor target loci present in the collection of DNA.

An additional embodiment of the present invention is the cloning of DNA fragment collections containing transcription factor target genes into bacteriophage arms and subsequent packaging into particles for the purposes of rapid conventional screening and sequencing. These bacteriophage libraries may be screened with known DNA probes or other unknown probes for purposes of discovery of target loci.

Yet another embodiment of the present invention is the cloning of DNA fragment collections containing transcription factor target genes into exon scanning vectors which may be introduced into eukaryotic cells for purposes of rapidly identifying potential coding sequences within the collection of DNA fragments.

Another embodiment of the present invention includes the nucleotide sequences and corresponding amino acid sequences and protein products as determined to be targets for either direct or indirect transcriptional regulation.

An additional embodiment of the present invention is the organization of the nucleotide and corresponding amino acid sequences discovered into a database or databases for purposes of rapid search and characterization of these sequences for functional and possible therapeutic relevance.

### 4.0 DESCRIPTION OF THE FIGURES

FIG. 1 Is a diagrammatic representation of transcriptional regulation by a steroid receptor transcription factor (see text for details).

FIG. 2 Is an illustration of the chemistry behind in vivo formaldehyde crosslinkage of nuclear protein/DNA interactions (see text for details).

FIG. 3 Is a diagrammatic illustration of the use of antibody-coated magnetic beads for the recovery of protein/DNA fragments (see text for details).

FIG. 4 Is a demonstration of the generation of “customizable” fragment sizes by adjustment of sonication conditions (see text for details).

FIG. 5 Is an outline of the technology described in the present invention for purposes of discovering transcription factor target genes (see text for details).

FIG. 6 Is a diagrammatic illustration of Exon Scanning.

FIG. 7A-D Is a demonstration of the utility of the described technology and invention through the analysis of RNA Polymerase II presence on the Sciara coprophila gene II/9-1 under different conditions (see text for details).

FIG. 8 Is a further demonstration of the utility of the described technology and invention and demonstrates p53 target gene identification after RNA Polymerase II large subunit “preIP/IP”, p53IP and stringent washing conditions (see text for details).

FIG. 9 Is a diagrammatic illustration of inverse PCR (I-PCR) applied towards DNA fragments isolated by methods described herein (see text for details).

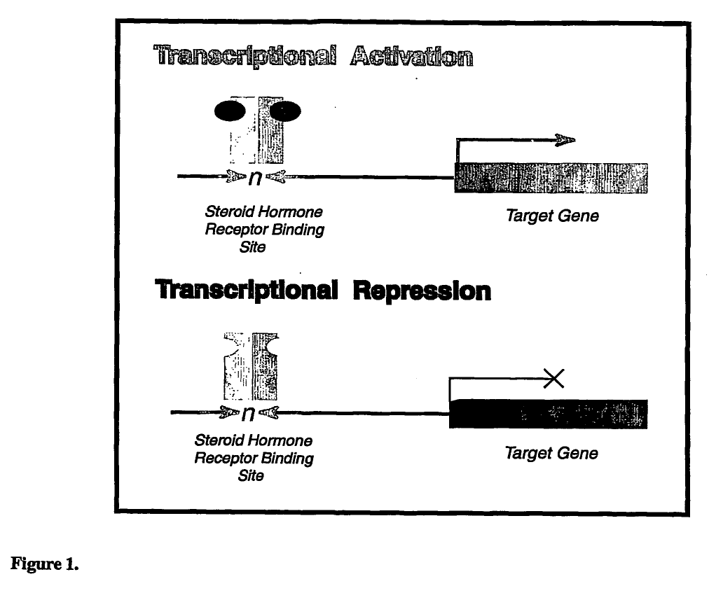

Table 1 Is a listing of two target nucleotide sequences representing regulatory elements identified for the transcription factor p53 and the relative induction of transcription from these sequences linked to a minimal promoter in the presence of p53 (see text for details).

### 5.0 DETAILED DESCRIPTION OF THE INVENTION

The presently described invention details a methodology for the rapid high-throughput identification of transcription factor target genes. It is achieved through the implementation of solid phase sequential chromosomal immunoprecipitation utilizing antibodies to both tissue and cell-type restricted transcription factors and those of the basal core transcriptional machinery. It is the application of this sequential immunoprecipitation which allows for efficient extraction of protein/DNA, RNA/DNA and RNA/DNA/protein complexes from living cells and or tissues. Combined with the presently described standard as well as modified molecular cloning methodologies these techniques result in rapid and thorough identification and characterization of transcription factor target loci. Particularly, implementation of solid phase sequential chromosomal immunoprecipitation in combination with modified inverse polymerase chain reaction, exon scanning and cloning strategies allows for the identification of direct transcription factor target loci. Implementation of solid phase sequential chromosomal immunoprecipitation in combination with cDNA library and microarray hybridization technologies also allows for rapid identification of transcription factor target genes.

The utility of the presently described inventions lies in the rapid identification of transcription factor target genes of both a direct (i.e. binds the factor) and indirect (factor is recruited to the gene through other proteins) nature from a living cell line or tissue. Application of the presently described invention allows for the vast identification of target loci for virtually any transcription factor of either a DNA binding or nonDNA binding nature. It is accomplished through a standard fixation of chromatin in living material, such as cells in tissue culture or isolated tissues, followed by successive immunoprecipitations of extracted protein/DNA complexes with antibodies specific to both transcription factors of interest as well as antibodies specific to the proteins of the core transcriptional machinery. Typically, DNA isolated by these methodologies may then be subjected to various molecular biology procedures such as IPCR, cloning into exon-trapping vectors and/or screening against cDNA libraries or microarrays of known genes to determine the content of actively transcribed genes pulled down with antibodies against chosen transcription factors.

Antibodies contemplated by the present invention are utilized for the purposes of immunoprecipitating either DNA binding or nonDNA binding proteins and may be of monoclonal or polyclonal origin. These antibodies described herein are designed against full length proteins as well as against particular epitope amino acid subsets present within those proteins. The antibodies are of rabbit and goat origin, but may be produced through the immunization of any of a number of organisms typically used for research antibody production.

The solid phase technology contemplated by the present invention involves the use of magnetic beads. These beads are conjugated to antibodies which specifically recognize particular proteins recovered from living cells and tissues. The magnetic aspect of the bead allows for efficient separation of the bead/antibody/protein/DNA complex from nonspecific materials, including wash solutions, present in the reaction mixture. Other solid phase technologies contemplated by the present invention include sepharose or other solid matrices linked to protein A, protein G or directly conjugated to antibodies which recognize specifically chosen proteins present within living cells/tissues.

For the purposes of the present invention the act of immunoprecipitating a protein/DNA complex will involve the utilization of an antibody of either polyclonal or monoclonal origin to directly and specifically recognize, bind and extract a protein/DNA complex from a bulk population of cross-linked protein/DNA complexes. It is this immunoprecipative process which allows for the efficient isolation and ultimate characterization of transcription factor target genes.

Molecular biology procedures described in the present invention include use of the collection of DNA fragments potentially containing transcription factor target genes recovered after immunoprecipitation to screen cDNA and/or genomic libraries. Additional molecular biology procedures include cloning the collection of DNA fragments potentially containing transcription factor target sequences into bacteriophage arms or plasmids for efficient screening and or sequencing.

For purposes of the present invention the term “gene” will refer to any and all regions of the genome of all organisms which code for proteins. This definition will also include all control elements directly or indirectly associated with controlling the production of mRNA from the gene.

In addition, for the purposes of the present invention the term “control element” will refer to any regulatory element which dictates, controls or modulates the production of mRNA from the corresponding gene. The production of mRNA is presumed to occur, at least in part, through the binding of transcription factors.

For the purposes of the present invention the term “transcription factor” will refer to any protein which binds directly or indirectly to a control element present within a gene and dictates, controls or modulates either the production or inhibition of production of mRNA from that particular gene.

As well, for the purposes of the present invention the term “transcriptional activator” will refer to any protein which binds either directly to a DNA control element or indirectly to a DNA control element through other proteins and activates or drives the production of mRNA from the gene corresponding to that particular control element.

For the purposes of the present invention the term “transcriptional repressor” will pertain to any protein which actively downregulates and thereby represses the production of mRNA from a gene to levels below those naturally occurring in an in vivo setting or to undetectable levels.

Also for the purposes of the present invention, the term “transcriptional modulator” will refer to any protein which dictates, controls or modulates the production of mRNA from a gene.

A gene will be delineated as active and therefore “expressed” when a nucleotide sequence referred to as an activating element is present within the gene or in close proximity to the gene and drives the production of detectable levels of mRNA, presumably through the actions of a transcriptional activating factor or transcriptional modulator. A gene will be delineated as not expressed when mRNA cannot be detected, presumably due to the absence of control activating elements, due to the absence of transcriptional activators present on those elements or due to the presence of transcriptional repressors.

Finally, for the purposes of the present invention the term “active repression” will refer to the direct downregulation of a gene due to the presence of a silencing element within that gene or in close proximity to the gene, presumably through the binding at that particular silencing element or negative regulatory element of a transcriptional repressor.

5.1 Transcriptional Regulation and Human Physiology

With the recent enormous influx of genomic information into the scientific community inevitably comes questions about genetic hierarchies and ultimately gene function. How gene activity is regulated and in what context is as crucial to an understanding of our genetic makeup as the sequence itself. More importantly, the question “What genes are expressed or repressed with respect to physiology?” represents an important concern regarding the discovery and characterization of drug targets. It is clear that the regulation of transcription plays a critical role in a limitless array of physiological processes. For example, a number of transcription factors have previously been implicated as either protooncogenes or tumor suppressors, thus affecting cancer progression by promotion or inhibition of cellular growth and apoptosis (for review see Levine et al., Nature, 1991, 351: 453-456). The transcription factor p53 has been shown to play an indispensable role in the suppression of tumorigenesis and thus has become to be known as a tumor suppressor in its wild-type form (Seto et al., Proc. Natl. Acad. Sci. USA, 1992, 89: 12028-12032). The statistical predisposition to tumorigenesis correlating with mutations in p53 is staggering, with for example, approximately 75-80% of all colon carcinomas studied exhibiting a loss of both p53 alleles. Such a preponderance for cancer upon inactivation of p53 DNA binding function strongly suggests that downstream targets for p53 transcriptional control may potentially play a role in tumor suppression and represent potential avenues of therapeutic intervention. Indeed, several of the targets for direct regulation by p53 have been demonstrated to be involved in the arrest or downregulation of cell proliferation and/or cell death. Examples of these include mdm2 (Oliner et al., Nature, 1993, 362: 857-860), p21/WAF1 (E1-Deiry et al., Cell, 1993, 75: 817-825), hsp70 (Maehara et al., Oncology, 2000, 58: 144-151), cyclin E (Smith et al., Exp. Cell Res., 1997, 230: 61-68) and MDR1 (Achanzar et al., Toxicol. Appl. Pharmacol., 2000, 64: 291-330). As p53 binding sites have been mapped in each of these loci, excellent internal controls exist for monitoring the sensitivity and background issues critical to the success of the technology described herein.

The p53 DNA recognition site consists of a dimer of two ten-mers which exists very rarely within the mammalian genome, occurring only around 300 times in a genome of three billion nucleotides (El-Deiry et al., Nat. Genet., 1992, 1(1): 45-49). This rare occurrence of the regulatory site for p53 provides a valuable assessment of the efficiency of the technology presented described technology. Sequence information acquired from fragments immunoprecipitated can be scanned for the presence of this site and direct targets immediately identified while background is simultaneously assessed.

In addition to p53, other factors have also been implicated in the progression of cancer. The female sex steroid hormone, estrogen, is required for the development and progression of human breast cancer. To understand how endocrine therapy works and why tumors may become resistant to one therapy but not another, an understanding of the molecular mechanisms of estrogen receptor (ER) function and identification of molecular targets for ER are required. The ER is a nuclear protein that functions as a transcription factor to regulate expression of estrogen responsive genes (Tenbaum et al., Int. J. Biochem. Cell Biol., 1997, 29: 1325-1341). Some of these estrogen-regulated genes mediate growth and development of the mammary glands, and it is apparent that many are important for the effects of estrogen on tumor cell proliferation. After estrogen or an estrogen analog binds to ER, dimerization of the receptor is induced which then allows binding of the complex to estrogen-responsive elements (ERE), a region in the promoter of estrogen target genes. The binding of the ER dimer to this promoter region then facilitates transcription of that gene. Most endocrine therapies for breast cancer inhibit tumor formation by depriving the cell of estrogen or by blocking its receptor. Synthetic drugs like tamoxifen were first called antiestrogens because they bind ER and competitively block the effects of estrogen on tumor cell proliferation and on expression of certain genes. However, it is not surprising that administration of this drug can have a spectrum of effects, depending on species, tissue, cell or gene context (Kazelenellenbogen et al., Breast Cancer Res. Treat., 1997, 44: 23-38.). In some cases, these “antiestrogens” can be estrogenic, stimulating transcription of genes which may change cellular morphology. For instance, tamoxifen, which works as an antagonist to ER in breast cancer cells, can induce tumor development in the uterus (Deligdisch, L., Mod. Pathol., 1993, 6(1): 94-106). In other cases, sometimes in the same cell, they have predominant antiestrogenic activity. These data provide the rationale for the identification of patterns for ER gene targets which can be activated and/or repressed upon the variety of drug treatments in different organs or tissues. Those molecular targets could potentially be used as important tumor markers and/or could provide additional indispensable information on hormonal responsiveness and further therapeutic intervention.

Determination of cell fate and regulation of terminal differentiation by transcription factors represent major roles for these regulatory proteins in regulating physiology. The ability to generate mature lymphocytic cells in tissue culture, for example, has been of intense interest for a number of years as the potential for replenishing low T and B cell counts in patients undergoing chemotherapy or are HIV positive, for example, becomes a reality. The ikaros family of transcription factors has been shown to promote the differentiation of hematopoietic stem cells into the mature B and T cell lineages (Nichogiannopoulou et al., Semin. Immunol., 1998, 10: 119-125). Correspondingly, mice which possess a mutation in the conserved DNA binding domain of the ikaros locus fail to possess B and T lymphocytes as well as the earliest progenitors of these lineages (Winandy et al., Cell, 1995, 83: 289-299). Thus, the ability to determine the downstream targets for ikaros allows for the potential to identify genes which promote hematopoietic stem cell differentiation and hence B and T cell production. The DNA recognition sequence for the ikaros family has been previously characterized (Molhar et al., Mol. Cell Biol., 1999, 14: 8292-8303), thus loci identified through the technology described herein as potential targets can be scanned for this recognition sequence as a confirmation of interaction.

Other more organogenic effects on human physiology are also controlled by transcription factors. Cardiac hypertrophy, or enlargement of the heart, is the result of attempts by the cardiovascular system to compensate for progression of many forms of cardiac disease, including hypertension, mechanical load, heart attack (myocardial infarction) and others (for review see McKinsey et al., Curr. Opin. Genet. Dev., 1999, 9: 267-274). At the molecular-level, external stress factors such as hypertension and myocardial infarction result in a reactivation of the fetal cardiac genetic program, as well as a general physiological enlargement of the myocardium through increased myocardial cell size. A number of transcription factors have been suggested to be involved in the initiation and maintenance of the reactivation of fetal cardiac genes. GATA4, a member of the GATA family of transcription factors, is involved in the upregulation of several fetal cardiac (Herzig et al., Proc. Natl. Acad. Sci. USA, 1997, 94: 7543-7348). Studies of GATA4 and other factors involved in response to cardiac stress will reveal novel cascades of genes representing potential targets for therapeutic prevention and/or intervention of enlargement of the heart.

In addition, there are a number of human genetic disorders affecting both growth and reproductive capacity. Several of these include mutations in transcription factors which have previously been shown to play vital roles in neuroendocrine organogenesis during embryonic development as well as appropriate functioning of this system in the adult (for review see Treier et al., Genes Dev., 1998, 12: 1691-1704). Defects in human growth and fertility represent a major concern among the world population. Many of the problems relating to these phenomena arise due to misregulation of genes which play crucial roles in the neuroendocrine system. Progress in understanding this complex field has been aided by the fact that several murine animal models have been shown to exhibit phenotypes strikingly similar to that demonstrated by allelic mutations in humans. For example, mutations in the human Prop-1 locus result in familial combined pituitary hormone deficiency, a finding quite similar to that found in the naturally occurring Ames mouse mutant (Wu et al., Nat. Genet., 1998, 6: 1143-1152). As well, both the Snell and Jackson dwarf mice have been shown to contain mutations within the Pit-1 locus (Rhodes et al., Curr. Opin. Genet. Dev., 1994, 4: 709-717). A number of human dwarfism cases which display similar pituitary lineage loss have now been demonstrated to carry mutations in the same locus (Wu et al., Nat. Genet., 1998, 6: 143-1152).

Both the Prop-1 and Pit-1 genes are POU domain-containing homeobox transcription factors which act at distinct temporal and spatial points within the development of the pituitary gland. Studies on the Ames dwarf have suggested that Prop-1 acts upstream of Pit-1 in the developmental regulatory cascade, putatively setting up a rudimentary organ from which Pit-1 is able to guide lineage determination and differentiation (Dasen et al., Cell, 1999,97: 587-598). Indeed, Pit-1 has been shown by a number of groups to play an indispensable role in the survival and terminal differentiation of the somatotrope, lactotrope and thyrotrope pituitary cell lineages (Rhodes et al., Curr. Opin. Genet. Dev., 1994, 4: 709-717). In the absence of these cell populations, specifically that of the somatotrophic lineage, dwarfism and other growth defects occur (Treier et al., Genes Dev., 1998, 12: 1691-1704). Many of the mutations which have been characterized in humans as well as other organisms for these factors lie in the DNA binding domain, which strongly suggests that an inability to effectively bind to and thus regulate downstream target genes is directly involved in the growth and fertility defects observed. An application of the technology described herein to identify and characterize both direct and indirect targets for these factors will undoubtedly reveal novel pathways for potential therapeutic intervention for both growth and fertility defects in humans.

It is evident that the activation or repression of gene activity is essential for the appropriate development, growth and viability of an organism. An understanding of the transcription factors described above as well as many others that govern these processes and specifically the identification of which target genes are controlled by these factors in both temporal and spatial manners during embryonic development and throughout adulthood is crucial to understanding various phenotypic characteristics. Current technologies such as subtractive hybridization (Lockyer et al., Parasitology, 2000, 120: 399-407), differential display (Neilson et al., Genomics, 2000, 3: 13-24) and SAGE (Stephan et al., Mol. Gen. Metab., 2000, 70: 10-18), while effective at identifying target genes, are generally time consuming and do not implicitly arrive at direct transcription factor targets. In addition, they require cell lines or tissues to differentially express the factor being studied, a task not often easily achieved. Other methods such as protein/DNA affinity purification may deduce enhancer binding sites from the genome but lack the ability to reveal the gene regulated by the enhancer, thus requiring positional cloning of exonic coding sequences (Solomon et al., Cell, 1988, 53: 937-947). Recent progress has been made, however, in the identification transcription factor targets and their corresponding coding sequences in vivo through the infection of living cells with modified retroviruses which seek out genomic transcription factor binding sites via integrase/transcription factor fusion proteins incorporated into the viral particle and “trap” exons (Burgess et al., U.S. Pat. No. 6,139,833, Issued Oct. 31, 2000).

By studying the intricate outline of terminal target genes regulated by various transcription factors rather than the factors themselves, it will be possible not only to discover novel therapeutic targets, but also to efficiently focus drug delivery to discrete physiologic gene products thus enhancing effectiveness and reducing or eliminating side effects. Moreover, the genes discovered as regulated by transcription factors may be used in a microarray format as phenotypical markers for medical diagnostics.

5.2 Modified-Chromosomal Immunoprecipitation

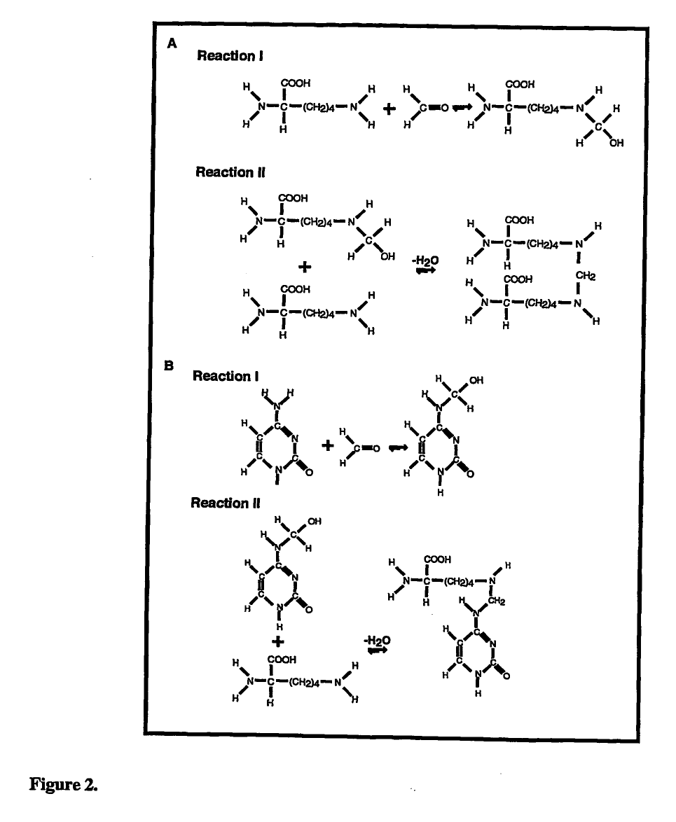

In order to identify and characterize direct molecular targets for regulation by specific transcription factors, it is necessary to employ technologies which take advantage of the extremely specific protein/DNA contacts involved in gene regulation which are maintained within intact cells or tissues. The Chromosomal Immunoprecipitation (ChIP) assay has been well established and may be successfully performed by those skilled in the art (Solomon et al., Cell, 1988, 53: 937-947; de Belle et al., Biotechniques, 2000, 29(19): 170-175). It allows for manipulation of the above mentioned inherent physical interactions between proteins and DNA to delineate known downstream targets for virtually any transcription factor. This method is based on the ability of formaldehyde or other chemicals to produce DNA/protein, RNA/protein and protein/protein cross-links at 2 angstrom resolution in vivo within intact cells or tissues. Addition of formaldehyde to living cells results in formation of an extensively cross-linked network of biopolymers, thus preventing any large-scale redistribution of cellular components. Formaldehyde does not react with free double-stranded DNA, avoiding kinetic constraints due to DNA damage. In addition, formaldehyde crosslinks can be reversed under mild conditions so that DNA, RNA and protein complexes can be further analyzed separately. FIG. 2 illustrates the chemistry behind the crosslinkage method. The experimental design originates from the pioneering work of Alexander Varshavsky who developed the chromatin fixation, purification and immunoprecipitation scheme for analyzing the distribution of histones in the Drosophila heat-shock gene promoter (Solomon et al., Cell, 1988, 53: 937-947). Upon reversal of crosslinkage and mechanical shearing of cellular DNA, protein/DNA interactions can be assessed by utilizing sequence information of known target loci in combination with the Polymerase Chain Reaction (PCR) (Innis et al., Academic Press, 1990; McPherson et al., IRL Press, 1991; Erlich, A. Stockton Press, 1989). Recent work has demonstrated that the ChIP assay can be applied to the study of virtually any transcription factor which comes into contact, either directly or indirectly, with DNA (Scully et al., Science, 2000, 290(5494):1127-31; Jepsen et al., Cell, 2000, 102(6):753-63).

Living cells and/or isolated tissues are fixed with formaldehyde by adding cross-linking agent directly to the cell growth medium or tissue. Although the presently described invention utilizes salivary glands from Sciara coprophila for RNA Polymerase II and Hela cells for p53 it is in no way limited to these particular tissues and cell types. Other tissues from other organisms and species include, but are not limited to heart, brain, spleen, lung, liver, muscle, kidney, testis, ovary, gut, hypothalamus, pituitary, tooth bud, mesoderm, ectoderm, endoderm, neural tube, somite, smooth muscle, cardiac muscle, skeletal muscle and all embryonic tissues from all possible timepoints. Cell lines from which transcription factor target genes may be discovered via methodologies provided by the presently described invention include, but are in no way limited to 13C4 (mouse/mouse, hybrid, hybridoma), 143 B (human, bone, osteosarcoma), 2 BD4 E4 K99 (mouse/mouse, hybrid, hybridoma), 3 C9-D11-H11 (mouse/mouse, hybrid, hybridoma), 3 E 1 (mouse/mouse, hybrid, hybridoma), 34-5-8 S (mouse/mouse, hybrid, hybridoma), 3T3 (mouse, Swiss albino, embryo), 3T3 L1 (mouse, Swiss albino, embryo), 3T6 (mouse, Swiss albino, embryo), 5 C 9 (mouse/mouse, hybrid, hybridoma), 5G3 (hybrid, hybridoma), 6-23 (clone 6) (rat, thyroid, medullary, carcinoma), 7 D4 (mouse/rat, hybrid, hybridoma), 72 A1 (mouse/mouse, hybrid, hybridoma), 74-11-10 (mouse/mouse, hybrid, hybridoma), 74-12-4 (mouse/mouse, hybrid, hybridoma), 74-22-15 (mouse/mouse, hybrid, hybridoma), 74-9-3 (mouse/mouse, hybrid, B cells x myeloma, hybridoma, B cell), 76-74 (mouse/mouse, hybrid, hybridoma), 7C2C5C12 (mouse/mouse, hybrid, B cells x myeloma, hybridoma), 9 BG 5 (mouse/mouse, hybrid, hybridoma), 9-4-3 (mouse/mouse, hybrid, hybridoma), A 172 (human, glioblastoma), A 375 (human, malignant melanoma), A 72 (dog, golden retriever, connective, not defined tumor), A427 (human, Caucasian, lung, carcinoma), A498 (human, kidney, carcinoma), A-704 (human, kidney, adenocarcinoma), A549 (human, lung, carcinoma), ACHN (human, Caucasian, kidney, adenocarcinoma), ACT 1 (mouse/mouse, hybrid, hybridoma), AE-1 (mouse/mouse, hybrid, hybridoma), AE-2 (mouse/mouse, hybrid, hybridoma), Aedes albopictus (mosquito—Aedes albopictus, larvae), AGS (human, Caucasian, stomach, adenocarcinoma), AK-D (cat, lung, embryonic), Amdur II human, Caucasian, skin, fibroblast, methylmalonicacidemia), AV 3 (human, amnion), B 95.8 (monkey, marmoset, leukocyte), B-63 (mouse, mammary gland, carcinoma), B2-1 (mouse, BALB/c, embryo), B50 (rat, nervous system, nervous tissue glial tumor), B69 (mouse/mouse, hybrid, hybridoma), B95a (monkey, marmoset), BAE (bovine, aorta), BALB 3T12-3 (mouse, BALB/c, embryo), BALB 3T3 clone A31 (mouse, BALB/c, embryo), BB (fish—Ictalurus nebulosus (bullhead brown catfish), trunk), BBM.1 clone E9 (mouse/mouse, hybrid, hybridoma), BC3H1 (mouse, brain, brain tumor), BCE C/D-1b (bovine, cornea), BeWo (human, placenta, choriocarcinoma), BF-2 (fish—bluegill fry, caudal trunk), BGM (monkey, African green, kidney), BHK 21 clone 13 (hamster, golden Syrian, kidney), BNL CL.2 (mouse, BALB/c, liver, embryonic), BNL SV A.8 (mouse, liver, embryonic), BS/BEK (bovine, kidney, embryonic), BSC-1 (monkey, African green, kidney), BT (bovine, turbinate), Bu (IMR-31) (buffalo, lung), BUD-8 (human, Caucasian, skin, fibroblast), BXPC-3 (human, pancreas, adenocarcinoma), C 1271 (mouse, RIII, mammary gland, mammary tumor), C2C12 (mouse, muscle), C32 (human, melanoma, amelanotic), C6 (rat, glial tumor), Caco-2 (human, Caucasian, colon, adenocarcinoma), Caki-1 (human, Caucasian, kidney, carcinoma), Caki-2 (human, Caucasian, kidney, carcinoma), CaLu-1 (human, Caucasian, lung, carcinoma, epidermoid), Calu-3 (human, Caucasian, lung, adenocarcinoma), CAPAN 1 (human, Caucasian, pancreas, adenocarcinoma), CAPAN 2 (human, Caucasian, pancreas, carcinoma), CAR (fish—goldfish, fin), CCF-STTRG1 (human, Caucasian, astrocytoma, anaplastic, grade IV), CCRF S 180 II (mouse, CFW, sarcoma), CCRF-CEM (human, Caucasian, peripheral blood, leukemia, acute lymphoblastic), CCRF-SB (human, Caucasian, peripheral blood, leukemia, acute lymphoblastic), CEM/C2 (human, leukemia, T cell), Cf2Th (dog, thymus), Chang liver (human, liver), CHO K1 (hamster, Chinese, ovary), CHP 3 (human, Black, skin, fibroblast, galactosemia), CHP 4 (human, Black, skin, fibroblast, asymptomatic galactosemia), CHSE 214 (fish—salmon, embryo), Clone 1-5c4 WKD of Chang Conjunctiva (human, conjunctiva), Clone M-3 (mouse, (CxDBA) F1, skin, melanoma), CMT 93 (mouse, C57BL/ICRFat, rectum, carcinoma), COS-1 (monkey, African green, kidney), COS-7 (monkey, African green, kidney), CPA (bovine, endothelium, pulmonary artery), CPA 47 (bovine, endothelium, pulmonary artery), CPAE (bovine, endothelium, pulmonary artery), CRFK (cat, domestic, kidney), CRI-D11 (rat, NEDH, insulinoma), CSE 119 (fish—salmon, embryo), CV 1 (monkey, African green, kidney), CVC 7 (Agrothis segetum, hybrid, hybridoma), D 17 (dog, bone, sarcoma, osteogenic), Daudi (human, Black, lymphoma, Burkitt), DB 9 G.8 (mouse/mouse, hybrid, hybridoma), DB 1-Tes (dolphin, Delphinus bairdi, testis), DeDe (hamster, Chinese, lung), Detroit 510 (human, Caucasian, skin, fibroblast, galactosemia), Detroit 525 (human, Caucasian, skin, fibroblast, Turner syndrome), Detroit 529 (human, Caucasian, skin, fibroblast, trisomy 21/Down syndrome), Detroit 532 (human, Caucasian, foreskin, trisomy 21/Down syndrome), Detroit 539 (human, Caucasian, skin, fibroblast, trisomy 21/Down syndrome), Detroit 548 (human, Caucasian, skin, fibroblast, partial D trisomy), Detroit 550 (human, skin, fibroblast), Detroit 551 (human, Caucasian, skin, embryonic), Detroit 562 (human, Caucasian, pharynx, carcinoma), Detroit 573 (human, Caucasian, skin, fibroblast, B/D translocation), Detroit 6 (human, bone marrow), DK (dog, beagle, kidney), DON (hamster, Chinese, lung), DU 145 (human, Caucasian, prostate, carcinoma), Duck embryo (duck, Pekin, embryo), E.Derm (horse, dermis), EBTr (bovine, trachea, embryonic), ECTC (bovine, thyroid, embryonic), ECV304 (human, Asiatic, umbilical cord), E[AV 12E8.1 (mouse/mouse, hybrid, hybridoma), Ep 16 (mouse/mouse, hybrid, hybridoma), EPC (fish, carp epidermal, epithelioma), EREp (rabbit, skin, embryonic), ESK-4 (pig, kidney, embryonic), FBHE (bovine, heart, embryonic), Fc 2 Lu (cat, lung, embryonic), Fc 3 Tg (cat, tongue, embryonic), FeLV 3281 (cat, lymphoma), FHM (fish—minnow, skin), FL (human, amnion), FRhK-4 (monkey, rhesus, kidney, embryonic), G-7 (mouse, Swiss-Webster, muscle), G.8 (mouse, Swiss-Webster, muscle), GCT (human, lung, metastasis, histiocytoma), GH 1 (rat, Wistar-Furth, pituitary tumor), GH 3 (rat, Wistar-Furth, pituitary tumor), Girardi heart (human, heart), GK 1.5 (mouse/rat, hybrid, hybridoma), H 16-L10-4R 5 (mouse/mouse, hybrid, hybridoma), H 9 (human, leukemia, acute lymphoblastic), H-4-II-E (rat, liver, hepatoma), H4 (human, Caucasian, brain, nervous tissue glial tumor), H4-II-E-C3 (rat, AxC, liver, hepatoma), H4TG (rat, liver, hepatoma), H9c2(2-1) (rat, BDIX, heart), Hak (hamster, Syrian, kidney), HCT 116 (human, colon, carcinoma), HCT-8 (human, intestine, ileocecal, adenocarcinoma), HEL 299 (human, Caucasian, lung, embryonic), HeLa (human, Black, cervix, carcinoma, epitheloid), HeLa 229 (human, Black, cervix, carcinoma, epitheloid), HeLa S 3 (human, Black, cervix, carcinoma, epitheloid), Hep 2 (human, Caucasian, larynx, carcinoma, epidermoid), Hep 3B2.1-7 (human, liver, carcinoma, hepatocellular), Hep G2 (human, Caucasian, liver, carcinoma, hepatocellular), Hepa 1-6 (mouse, liver, hepatoma), HFL (human, lung), HG 261 (human, Caucasian, skin, fibroblast, Fanconi anemia), HGF 24 (human, gingival stroma), HL 60 (human, Caucasian, peripheral blood, leukemia), HOS (human, Caucasian, bone, osteosarcoma), HRT 18 (human, rectum-anus, adenocarcinoma), Hs 683 (human, neuroglia, glioma), Hs 863.T (human, bone, sarcoma, Ewing's), HS 883.T (human, bone, giant cell, sarcoma), HS 888 Lu (human, Caucasian, lung), Hs-27 (human, foreskin), HSDM1C1 (mouse, Swiss albino, fibrosarcoma), HT 1080 (human, Caucasian, acetabulum, fibrosarcoma), HT 1376 (human, Caucasian, bladder, carcinoma), HT-29 (human, Caucasian, colon, adenocarcinoma), HuTu 80 (human, adenocarcinoma), I 10 (mouse, BALB/cJ, testis, Leydig cells, testicular tumor), IB-RS-2 (pig, kidney), IBRS-2 D10 (pig, kidney), IEC-6 (rat, intestine, small), IM-9 (human, Caucasian, bone marrow, multiple myeloma), IMR 31 Bu (buffalo, lung), IMR 32 (human, Caucasian, neuroblastoma), IMR-90 (human, Caucasian, lung, embryonic), Intestine 407 (human, Caucasian, intestine, embryonic), J 111 (human, leukemia, monocytic), J 774A. 1 (mouse, BALB/c, monocyte-macrophage, not defined tumor), Jensen sarcoma (rat, sarcoma), JH 4 clone 1 (guinea pig, strain 13, lung), Jiyoye (human, Black, ascitic fluid, lymphoma, Burkitt), JM (human, leukemia, T cell), Jurkat J6 (human, leukemia, T cell), K 562 (human, Caucasian, pleural effusion, leukemia, chronic myeloid), KATO III° (human, Mongoloid, stomach, carcinoma), KB (human, Caucasian, mouth, carcinoma, squamous cell), KHOS/NP (human, Caucasian, bone, osteosarcoma), KMP (mouse), L 1210 (mouse, ascitic fluid, leukemia, lymphocytic), L 132 (human, lung, embryonic), L 21.6 (mouse, hybrid, hybridoma), L 243 (mouse/mouse, hybrid, hybridoma), L 5.1 (mouse/mouse, hybrid, hybridoma), L 929 (mouse, C3H/An, connective), L6 (rat, skeletal muscle), LC 540 (rat, Fisher, testis, Leydig cells, testicular tumor), LLC-MK2 (monkey, rhesus, kidney), LLC-PK1 (pig, kidney), LLC-RK1 (rabbit, New Zealand white, kidney), LLC-WRC 256 (rat, Walker, carcinoma), LM from NCTC clone 929 (mouse, C3H/An, connective), LM TK negative (mouse, C3H/An, connective), LNCaP.FGC (human, Caucasian, prostate, carcinoma), LS 180 (human, Caucasian, colon, adenocarcinoma), M 1 (mouse, SL, bone marrow, leukemia, myeloid), M-2E6 (mouse/mouse, hybrid, hybridoma), M2-1C6-4R3 (mouse/mouse, hybrid, hybridoma), MA 104 (monkey, African green, kidney, embryonic), mAB 35 (mouse/rat, hybrid, B cells x myeloma, hybridoma, B cell), MARC 145 (monkey, kidney), Mc Coy (mouse), MC/CAR (human, plasmacytoma, B cell), MCF 7 (human, Caucasian, breast, adenocarcinoma), MDBK (bovine, kidney), MDBK(BU 100) (bovine, kidney), MDCC MSB1 (chicken, avian, spleen, lymphoma), MDCK (dog, cocker spaniel, kidney), MDOK (sheep, kidney), MDTC RP 19 (turkey, lymphocyte, Marek's disease), MEL III (monkey, rhesus, mammary gland, mammary tumor), MG-63 (human, bone, osteosarcoma), MH 1 C 1 (rat, buffalo, liver, hepatoma), MH-S (mouse, lung), MIA PaCa-2 (human, Caucasian, pancreas, carcinoma), MiCl1 (mustela vison (mink), lung), MK-D6 (mouse/mouse, hybrid, hybridoma), MLA 144 (gibbon, lymphosarcoma), MOLT-3 (human, peripheral blood, leukemia, acute lymphoblastic T cell), MOLT-4 (human, peripheral blood, leukemia), MPC-11 (mouse, BALB/c, myeloma), MPK (minipig, kidney), MRC-5 (human, lung, embryonic), MRSS-1 (mouse/mouse, hybrid, hybridoma, B cell), MS (monkey), Mv 1 Lu (mustela vison (mink), lung), MVPK-1 (pig, kidney), NA C 1300 clone (mouse, brain, neuroblastoma), Namalwa (human, Black, lymphoma, Burkitt), NCTC 2544 (human, skin, keratinocyte), NCTC clone 3526 (monkey, rhesus, kidney), Neuro-2a (mouse, albino, neuroblastoma), NIH:OVCAR-3 (human, Caucasian, adenocarcinoma, ovary), NOR 10 (mouse, muscle), NRK 49F (rat, kidney), NSO (mouse, BALB/c, myeloma), OA1 (sheep, brain), OHH1.K (deer, kidney), OKT 3 (mouse/mouse, hybrid, hybridoma), OKT 4 (mouse/mouse, hybrid, hybridoma), OKT 8 (mouse/mouse, hybrid, hybridoma), P 3 HR 1 (human, lymphoma, Burkitt), P3 88 D1 (mouse, DBA/2, monocyte-macrophage, lymphoma), P3 NS1 Ag4 (mouse, myeloma), P3NP/PFN (mouse/mouse, hybrid, hybridoma), P815 (mouse, mastocytoma), PANC-1 (human, Caucasian, pancreas, carcinoma), PC 61-5-3 (mouse/rat, hybrid, hybridoma), PC-12 (rat, adrenal medulla, pheochromocytoma), PD 5 (pig, kidney), PEG 1-6 (mouse/mouse, hybrid, B cells x myeloma, hybridoma, B cell), PK 15 (pig, kidney), PLC/PRF/5 (human, liver, hepatoma, Alexander cells), Pt K1 (marsupial—potoroo, kidney), QT35 (quail, Japanese, fibrosarcoma), QT 6 (quail, Japanese, fibrosarcoma), R 2 C (rat, Wistar-Furth, testis, Leydig cells, testicular tumor), R 9 ab (rabbit, New Zealand white, lung), R D (human, Caucasian, muscle, rhabdomyosarcoma, embryonal), R63 (mouse/mouse, hybrid, B cells x myeloma, hybridoma, B cell), RAB-9 (rabbit, New Zealand white, skin, fibroblast), Raji (human, Black, lymphoma, Burkitt), RBL 1 (rat, leukemia, basophilic), RFL 6 (rat, Sprague-Dawley, lung), RK 13 (rabbit, kidney), RK 13/1 (rabbit, kidney), RPMI 1788 (human, Caucasian, peripheral blood), RPMI 1846 (hamster, golden Syrian, skin, melanoma, melanotic), RPMI 2650 (human, nasal septum, carcinoma, squamous cell), RPMI 8226 (human, peripheral blood, myeloma), RR 1022 (rat, Amsterdam, sarcoma), RTG 2 (fish—trout, rainbow, gonad), RTO (fish—trout, rainbow, ovary), Saos-2 human, Caucasian, bone, osteosarcoma), Sf 1 Ep (rabbit, domestic, epidermis), SIRC (rabbit, cornea), SK-LU-1 (human, Caucasian, lung, adenocarcinoma, grade III), SK-MES-1 (human, lung, carcinoma, squamous cell), SK-NEP-1 (human, Caucasian, kidney, Wilms' tumor), SK-OV-3 (human, Caucasian, ovary, adenocarcinoma), SSE 5 (fish—trout, embryo), STO (mouse, SIM, embryo), SV-T2 (mouse, BALB/c, embryo), SW 13 (human, Caucasian, adrenal cortex, adenocarcinoma), T 98 G (human, Caucasian, glioblastoma), Tb 1 Lu (bat, lung), TE 671 (human, Caucasian, medulloblastoma), TK TS 13 (hamster, Syrian, kidney), U 937 (human, Caucasian, pleural effusion, lymphoma, histiocytic), VERO (monkey, African green, kidney), VERO 76 (monkey, African green, kidney), VERO C 1008 (monkey, African green, kidney), WC 1 (fish, dermis, sarcoma), WF 2 (fish—Walley whole fry, fibroblast), WI 26 VA 4 (human, Caucasian, lung, embryonic), WI 38 (human, Caucasian, lung, embryonic), WI 38 VA 13 (human, Caucasian, lung, embryonic), WI-1003 (human, lung), WISH (human, amnion), WM 115 (human, skin, melanoma), XC (rat, Wistar, sarcoma), Y 1 (mouse, LAF1, adrenal cortex, adrenal tumor), ZR-75-1 (human, Caucasian, breast, carcinoma) and any other as yet undiscovered or uncharacterized cell lines through which the presently described invention may be implemented for the discovery of transcription factor target genes.

Preliminary time course experiments spanning between 5 minutes and 1 hour of fixation are performed to yield the best combination of in vivo fixed chromatin, high DNA recovery, and small size of chromatin fragments. For specific purposes, the cross-linking time can be considerably reduced or prolonged and must be optimized for the particular tissue or cell line and the transcription factors being studied. FIG. 2 illustrates the chemical cross-linking of DNA and proteins by formaldehyde. Formaldehyde (HCHO) is a very reactive dipolar compound in which the carbon atom is the nucleophilic center. Amino and imino groups of the proteins (e.g. the side chains of lysine and arginine) and of nucleic acids (e.g., cytosines) react with formaldehyde, leading to the formation of a Schiff base (reaction I). This intermediate can react with a second amino group (reaction II) and condenses. Cross-links may be reversed by heating in Tris-HCL—containing buffers. This leads to a drop in pH and protonation of amino groups, thus forcing the equilibrium in the reverse direction. In FIG. 2(A) illustrates formaldehyde-mediated cross-linking between the side chains of the lysines and (B) depicts cross-linking between cytosine and lysine.

While the present invention employs formaldehyde as a chemical component for the cross-linking of protein/DNA complexes in living cells and tissues, it is in no way limited to this reagent for fixation. Other chemicals may also be utilized to fix proteins to DNA (Benashski et al., Methods, 2000, 22: 365-371). Some of these include, but are in no way limited to homobifunctional compounds difluoro-2,4-nitrobenzene (DFDNB), dimethyl pimelimidate (DMP), disuccinimidyl suberate (DSS), thcarbodimide reagent EDC, psoralens including 4,5′, 8-trimethylpsoralen, photo-activatable azides such as ¹²⁵I(S-[2-(4-azidosalicylamido)ethylthio]-2-thiopyridine) otherwise known as AET, (N-[4-axidosalicylamido)butyl]-3′[2′-pridyldithio]propionamide) also known as APDP, the chemical cross-linking reagent Ni(II)-NH2-Gly-Gly-His-COOH also known as Ni-GGH, sulfosuccinimidyl 2-[(4-axidosalicyl)amino]ethyl]-1,3-dithiopropionate) also known as SASD, (N-14-(2-hydroxybenzoyl)-N-11(4-azidobenzoyl)-9-oxo-8,11,14-triaza-4,5-ditheatetradecanoate) and any as yet uncharacterized or undiscovered reagents which result in the cross-linking of protein/DNA complexes in living cells and tissues.

Upon fixation of protein/DNA complexes in intact cells or tissues cellular lysis is accomplished through standard protocols which may be successfully implemented by those skilled in the art (Solomon et al, Cell, 1988, 53: 937-947; de Belle et al., Biotechniques, 2000, 29(1): 170-175). For the purposes of chromosomal immunoprecipitation it is important that metal chelators such as EDTA and EGTA as well as protease inhibitors be added to the reaction to prevent degradation of protein/DNA complexes. The mixture is subsequently mechanically lysed on ice via the its passage through a 26 G needle. Typically 4 rounds of 25-30 needle passages per round are necessary for sufficient lysis and chromatin fractionation. It is speculated that these parameters must be defined for each tissue or cell type. Alternatively, samples may be lysed via the use of a Dounce homogenizer or the implementation of any mechanical stress which results in efficient breakage of cellular membranes and hence release of chromatin containing protein/DNA complexes.

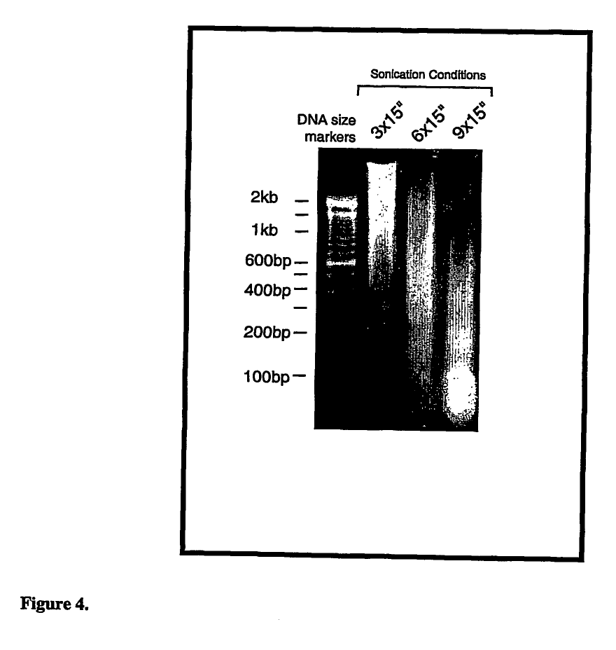

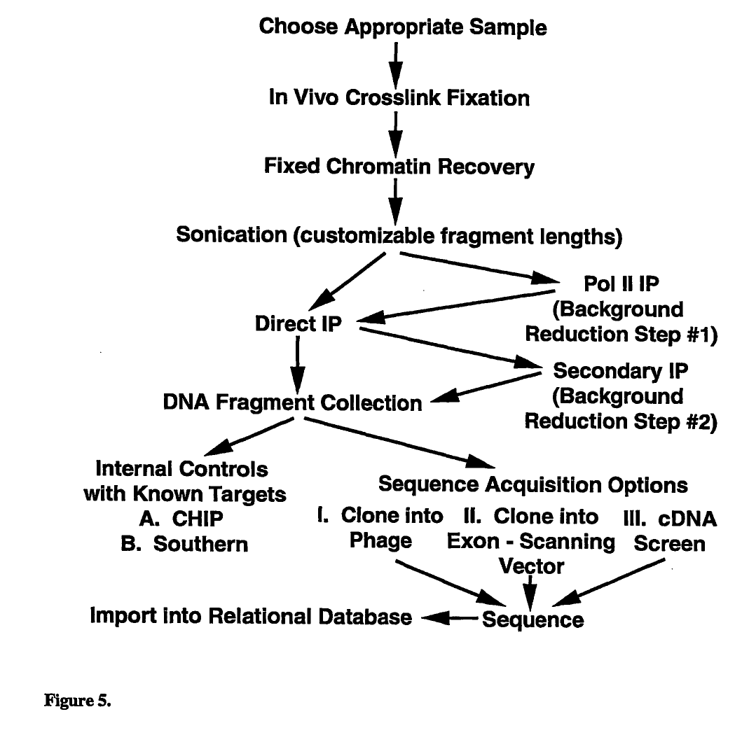

Fixed, lysed cells or tissues are subsequently subjected to high resolution mechanical shearing, i.e. sonication for the purposes of producing manageable DNA fragment sizes of the desired length. In general, the size of the DNA fragments may be critical for high-resolution mapping studies as well as the identification of transcription initiation sites and/or exonic sequences, thus sonication provides a convenient method to “customize” fragment length as illustrated in FIG. 4. The sizes observed are a typical result obtained with Hela cells cross-linked for 30 minutes and sonicated by routine use of the Branson model 250 sonifer with microtip at constant power for various amounts of time. While the presently described invention employs the use of a Branson model 250 sonifier/sonicator for the purposes of generating appropriate DNA fragment length from fixed lysed cells and/or tissues it is hypothesized that any mechanical instrument or enzymatic digestion capable of shearing or cutting soluble chromatin into lengths small enough to be manipulated via standard or modified molecular biology procedures for the purposes of discovering transcription factor targets may be utilized, These include, but are in no way limited to other sonicator models as well as restriction enzyme digestion by frequent as well as rare-cutting enzymes including, but in now way limited to, Acc I, Aci I, Acl I, Afe I, Afl II, Afl III Age I, Ahd I, Alu I, Alw I, AlwN I, Apa I, ApaL I, Apo I, Asc I, Ase I, Ava I, Ava II, Avr II, Bae I, BamH I, Ban I, Ban II, Bbs I, Bbv I, BbvC I, BceA I, Bcg I, BciV I, Bcl I, Bfa I, BfrB I, Bgl I, Bgl II, Blp I, Bmr I, Bpm I, BsaA I, BsaB I, BsaH I, Bsa I, BsaJ I, BsaW I, BsaX I, BseR I, Bsg I, BsiE I, BsiHKA I, BsiW I, Bsl I, BsmA I, BsmB I, BsmF I, Bsm I, BsoB I, Bsp1286 I, BspD I, BspE I, BspH I, BspM I, BsrB I, BsrD I, BsrF I, BsrG I, Bsr I, BssH II, BssK I, BssS I, BstAP I, BstB I, BstE II, BstF5 I, BstN I, BstU I, BstX I, BstY I, BstZ17 I, Bsu36 I, Btg I, Btr I, Bts I, Cac8 I, Cla I, Dde I, Dpn I, Dpn II, Dra I, Dra III, Drd I, Eae I, Eag I, Ear I, Eci I, EcoN I,EcoO109 I, EcoR I, EcoR V, Fau I, Fnu4H I, Fok I, Fse I, Fsp I, Hae II, Hae III, Hga I, Hha I, Hinc II, Hind III, Hinf I, HinP1 I, Hpa I, Hpa II, Hpy188 I, Hpy188 III, Hpy99 I, HpyCH4III, HpyCH4IV, HpyCH4V, Hph I, Kas I, Kpn I, Mbo I, Mbo II, Mfe I, Mlu I, Mly I, Mnl I, Msc I, Mse I, Msl I, MspA1 I, Msp I, Mwo I, Nae I, Nar I, Nci I, Nco I, Nde I, NgoM IV, Nhe I, Nla III, Nla IV, Not I, Nru I, Nsi I, Nsp I, Pac I, PaeR7 I, Pci I, Pf1F I, Pf1M I, Ple I, Pme I, Pml I, PpuM I, PshA I, Psi I, PspG I, PspOM I, Pst I, Pvu I, Pvu II, Rsa I, Rsr II, Sac I, Sac II, Sal I, Sap I, Sau3A I, Sau96 I, Sbf I, Sca I, Scrf I, SexA I, SfaN I, Sfc I, Sfi I, Sfo, SgrA I, Sma I, Sml I, SnaB I, Spe, Sph I, Ssp I, Stu I, Sty I Swa I, Taq I, Tfi I, Tli I, Tse I, Tsp45 I, Tsp509 I, TspR I, Tth111 I, Xba I, Xcm I, Xho I, Xma I, Xmn I and any other as yet uncharacterized or undiscovered restriction endonucleases which may be utilized to cut DNA for the purposes of implementing the presently described invention to discover transcription factor target genes of both known and unknown origin.

It is the ability to customize the length of said DNA fragments which allows for the cloning of transcription factor targets upon immunoprecipitation utilizing solid phase sequential immunoprecipitation. After fixation and subsequent sonication, fixed chromatin fragments of defined length binding the protein (i.e. transcription factor) of interest either directly or indirectly are purified by selective immunoprecipitation with antibodies specific to 1) proteins present within the core transcriptional machinery, an example of which is the large subunit (c) of RNA polymerase II and 2) the particular transcription factor for which target genes are being sought (see below for detailed description of this procedure). As discussed below, it is the solid phase sequential immunoprecipitation procedure utilizing antibodies to both the core transcriptional machinery proteins as well as specific transcription factors which allows for the efficient cloning and characterization of coding sequences for transcription factor target genes.

5.3 Multiple Rounds of Chromosomal Immunoprecipitation Reduce Background

While it is clear that it is possible to obtain known in vivo target loci for numerous transcription factors utilizing conventional chromosomal immunoprecipitation technologies, an inherent problem is the retrieval of nonspecific protein/DNA complexes. These false positives are often the result of interactions between proteins and noncoding, inactive genomic DNA. While often relevant, these interactions may be those which occur at great distances from the transcription initiation site and thus the identification of coding sequence for the target loci pertaining to these protein/DNA contacts becomes difficult. The presently described technology circumvents the issues of nonspecificity and regulatory element distance from the transcription initiation site through an immunoprecipitation step utilizing antibodies to components of the basal transcriptional machinery. As outlined in FIG. 5, chromatinized template is immunoprecipitated with antibodies specific for particular transcription factors. In order to enrich for loci actively regulated by these factors, the presently described invention describes preincubation of chromatinized templates with monoclonal antibodies specific for the large subunit (c) of RNA polymerase II (Background reduction step 1). This “preIP” immunoprecipitation enriches for genes actively transcribed by the Pol II transcription machinery thereby reducing the nonspecificity of the secondary immunoprecipitation and helps to overcome problems related to higher complexity of the genome by omitting noncoding regions and satellite DNA together with nontranscribed genes. Said “preIP” is performed via the solid phase sequential chromosomal immunoprecipitation protocol described herein and may be successfully implemented by those known and skilled in the art.

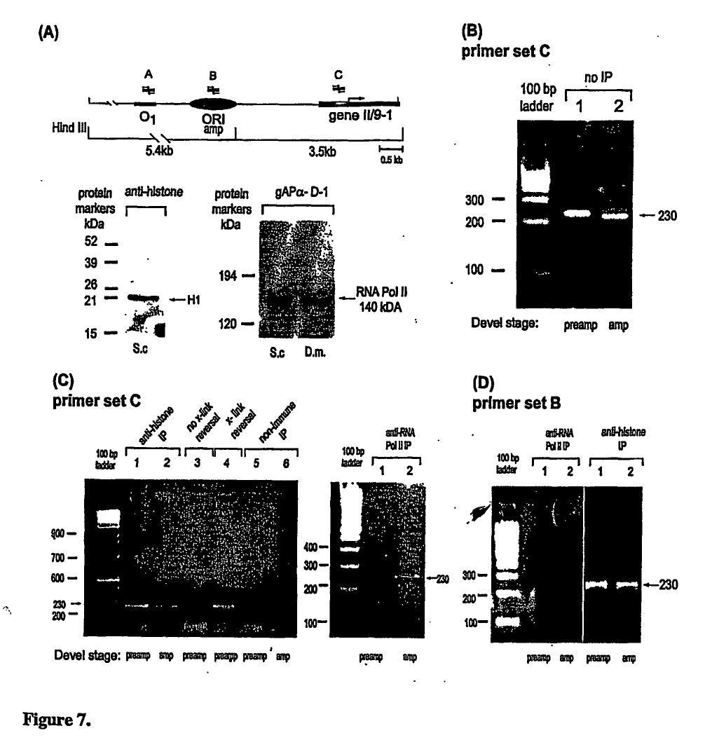

FIG. 7 demonstrates the utility of the presently described invention as it pertains to chromosomal immunoprecipitation with antibodies specific for core chromatin proteins and antibodies specific for the large subunit of RNA polymerase II of Sciara coprophila (see Example 6.1 for details and Weeks et al., Genes Dev., 1993, 7: 2329-2344). It illustrates the necessity of cross-linkage reversal as well as the customizable capability of sonication for the purposes of producing chromatin fragments which can be immunoprecipitated discretely with respect to core chromatin proteins or core transcriptional apparatus proteins. IgG antibodies utilized and contemplated by the present invention include those specific for the Drosophila melanogaster RNA Polymerase II large subunit. These antibodies also cross react with the large subunit of RNA Polymerase II from the fly species Sciara coprophila. Species of origin for these antibodies is goat. Termed gAP α-D1, the IgGs were affinity purified using a column carrying a fusion protein term D1, which contains residues A519-Gly992 of the IIc subunit. As well as cross-reacting with Sciara coprophila RNA Polymerase II, the antibodies mildly cross react with the large subunit of yeast as well as mammalian RNA Polymerase II (Weeks et al., Genes & Development, 1993, 7: 2329-2344). A 1:1000 dilution of the original stock solution of 22 ug IgG in 50 ul PBS was used. In addition, a second set of antibodies affinity purified from rabbit immunosera, termed rAP α-PCTD, recognizes the hyperphosphorylated C-terminal domain of Drosophila RNA Polymerase II. A dilution of 1:500 of an original stock solution of 0.054mg/ml in PBS/50% ethylene glycol was used. A third set of antibodies utilized in the presently described invention, termed gAP α-CTD, specifically recognizes the unphosphorylated C-terminal domain of Drosophila RNA polymerase II large subunit. A 1:2000 dilution of an original stock solution of 0.51mg/ml 2× PBS was used.

Other antibodies contemplated by the present invention include those designed to discrete regions of the RNA Polymerase II individual subunits including IIc. These antibodies may be of either monoclonal or polyclonal origin. Examples of these antibodies contemplated by the present invention include rabbit affinity purified polyclonal antibody specific for a peptide mapping within the tandem repeat domain of the large subunit of murine RNA Polymerase II. An additional antibody contemplated by the present invention includes an affinity purified rabbit polyclonal antibody raised against a peptide mapping to the amino terminus of the large subunit of RNA Polymerase II. Yet a third antibody contemplated by the present invention includes a rabbit polyclonal antibody raised against a recombinant protein corresponding to amino acid 1-224 of RNA Polymerase II of human origin (for review see Tjian, R. and Maniatis, T., Cell, 1994, 77: 5-8). The presently described invention covers any antibodies designed to interact with or bind specifically the large subunit of RNA polymerase II.

The presently described invention is in no way limited to utilization of the above antibodies for purposes of first-round immunoprecipitation. Additionally, antibodies to other proteins and subunits present within the core basal transcriptional machinery may be utilized. It is contemplated by the present invention that sequential chromosomal immunoprecipitation utilizing antibodies to any protein present within the core transcriptional apparatus may substantially increase the ability to identify transcribed regions of transcription factor target loci (Kuras et al., Science, 2000, 19: 1244-1248). Subunits of the core transcriptional apparatus, specifically that of the transcriptional initiation complex, for which chromosomal immunoprecipitation may be successfully carried out as discussed in the presently described invention include, but in no way are limited to species RNA polymerase IIA, RNA polymerase IIB and RNA polymerase IIc. Other antibodies contemplated by the present invention may be designed to bind specifically to other core transcriptional apparatus proteins exclusive of the large subunit of RNA polymerase II (Nikolov et al., Proc. Natl. Acad, Sci. USA, 1997, 94: 15-22; Hoffmann et al., Proc. Natl. Acad. Sci. USA, 1997, 94: 8928-8935). These include, but in no way are limited to TAF, TAF(I110), TAF(148), TAF(I63), TAF(II100), TAF(II110), TAF(II125), TAF(II135), TAF(II145), TAF(II150), TAF(II170), TAF(II18), TAF(II19), TAF(II20), TAF(II 25), TAF(II250), TAF(II25ODelta), TAF(II28), TAF(II30), TAF(II30alpha), TAF(II30beta), TAF(II31), TAP(II40), TAF(II47), TAF(II55), TAF(II60, TAF(II61), TAF(II67), TAF(II70-alpha), TAF(II70-beta), TAF(II70-gamma), TAF(II80), TAF-1, TAF-90, TAF-I, TAF-II, TAF-L, TBF1, TBP, TBP-1, TBP-2, TFIIA (32 kDa subunit, TFIIA-alpha/beta precursor (major), TFIlA-alpha/beta precursor (minor), TFIIA-gamma, TFIIA-L, TFIlA-S, TFIIB, TFIID, TFIIE, TFIIE-alpha, TFIIE-beta, TFIIF, TFIIF-alpha, TFIIF-beta, TFIIH, TFIIH core, TFIIH*,TFIIH-CAK, TFIIH-CCL1, TFIIH-cyclin H, TFIIH-ERCC2/CAK, TFIIH-KIN28, TFIIH-MAT1, TFIIH-MO15, TFIIH-p34, TFIIH-p37, TFIIH-p38, TFIIH-p44, TFIIH-p50, TFIIH-p55, TFIIH-p62, TFIIH-p73, TFIIH-p80, TFIIH-p⁸⁵, TFIIH-p90, TFIIH-SSL2/RAD25, TFIIH-TFIIK, TFII-I, TFIIIA and any other as yet uncharacterized or undiscovered proteins which interact with the core transcriptional machinery for purposes of initiating transcriptional activation. It should be noted that utilization of antibodies to these proteins may produce conflicting results as evidence exists that genes may be repressed even in the presence of core transcriptional machinery proteins, with the exception of the dephosphorylated form of the large subunit (c) of RNA polymerase II (Tjian and Maniatis, Cell, 1994, 77: 5-8). As mentioned above, other as yet undiscovered and thus undescribed basal transcriptional apparatus proteins exclusive of the large subunit of RNA polymerase II are also contemplated by the present invention for the purposes of carrying out sequential immunoprecipitation to identify actively transcribed transcription factor target loci.

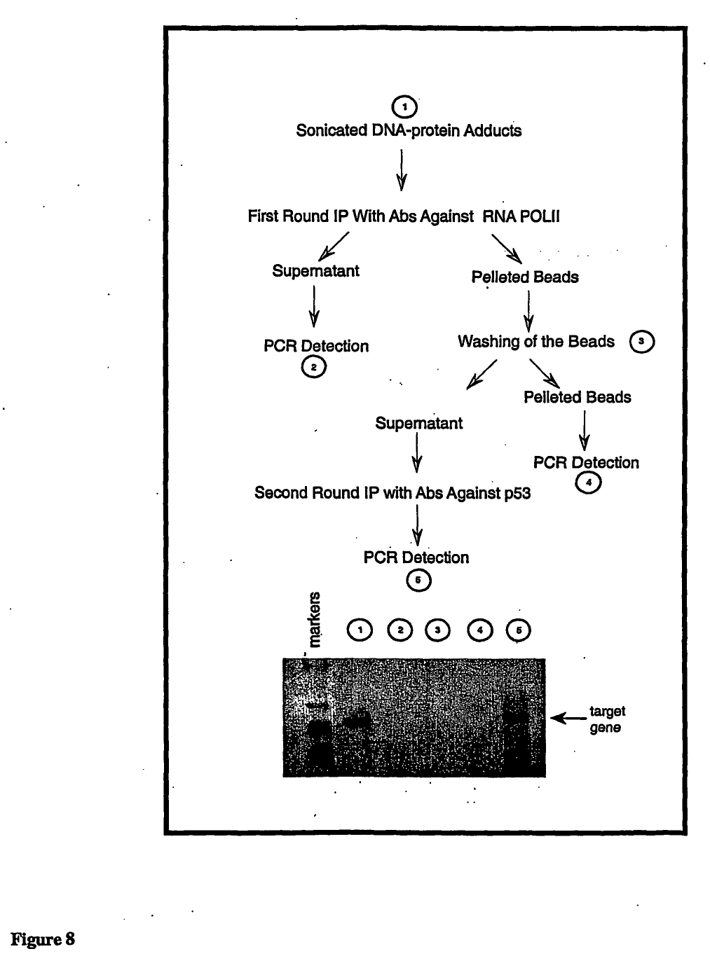

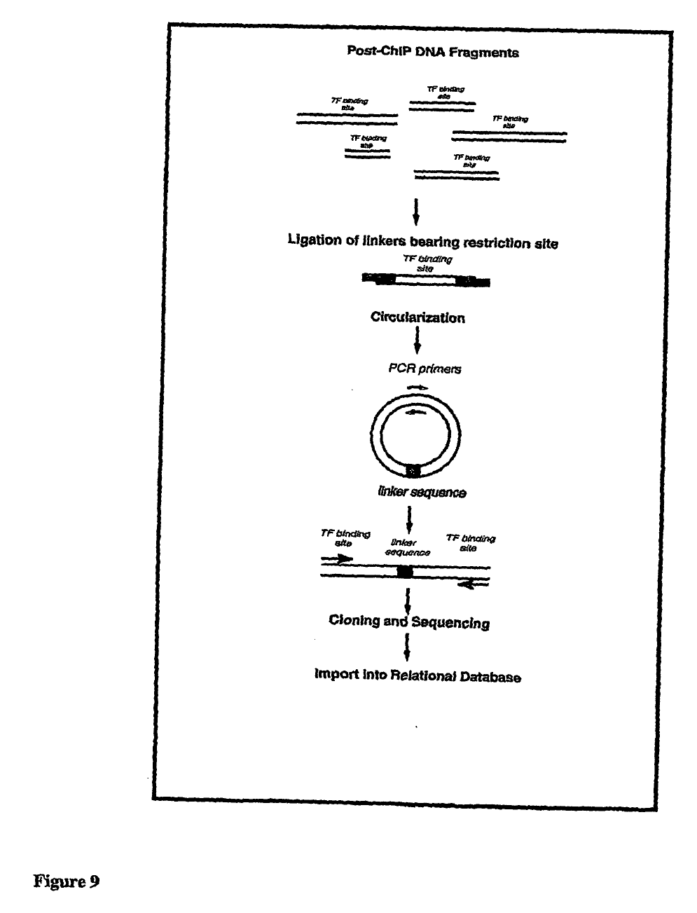

FIG. 8 demonstrates the utility of sequential immunoprecipitation for the purposes of identifying a known p53 target gene, p21. As is evidenced, very little quantitative PCR detection signal is lost due to sequential immunoprecipitation as compared to precipitation with antibodies only specific for the large subunit of RNA polymerase II (see the flowchart and lanes 1 through 4 which represent different stages of the sequential immunoprecipitation procedure for details). As mentioned below, the presently described invention employs the use of a solid phase support, in this case magnetic beads, for increasing the yield of immunoprecipitated cross-linked chromatin during the implementation of sequential chromosomal immunoprecipitation. In order to provide a cross-linked protein/DNA complex as a substrate for the second round of immunoprecipitation with an antibody specific for the particular transcription factor being studied, it is necessary to release previously bound solid phase support from the protein/DNA complex. This is accomplished in the presently described invention via pH alteration. By increasing the acidity of the complex mixture antibodies linked to the solid phase are denatured and bound cross-linked DNA/protein complexes are released. In the experiment described the pH was adjusted from a neutral value of pH 7.6 to an acidity of 5.5 for the efficient denaturation of antibodies covalently linked to the solid phase. pH alteration may be performed to successfully denature antibodies on the solid phase by those known and skilled in the art, but must be determined experimentally for each particular antibody and solid phase support utilized for sequential immunoprecipitation. It is the denaturation of antibodies linked to solid phase which allows for the release of cross-linked pull-down DNA/protein complexes and the next round of chromosomal immunoprecipitation to be carried out and is hence covered by the present invention. Other methods contemplated and covered by the present invention for the denaturation of antibodies bound to solid phase supports for the purposes of sequential immunoprecipitation include, but are in no way limited to enzymatic digestion including but not limited to proteolysis, temperature alteration, chemical, mechanical and UV dissociation. In addition, it is contemplated by the present invention that the junction between the antibody and its solid support matrix may also be manipulated by the above methods for removal of chromatin template for the purposes of second round immunoprecipitation.

Table 1 delineates the identification of two previously uncharacterized target regulatory elements for the transcription factor p53 discovered through utilization of technology described by the present invention. The nucleotide sequences listed demonstrate near consensus p53 binding sites and elicit a severalfold increase in stimulation in standard cotransfection induction experiments.

It is clear that by performing sequential immunoprecipitation utilizing antibodies specific to the large subunit (c) of RNA polymerase II only actively transcribed transcription factor target genes will be identified due to the required clearance of the promoter prior to large subunit attachment. It is possible, however, to also discover and identify genes which are actively repressed by transcription factors which beacon repressor molecules that inhibit promoter clearance. An example of such a repressor is NcoR (Heinzel et al., Nature, 1997, 387: 43-48). It is contemplated in the presently described invention that utilization of antibodies specific for NcoR in combination with antibodies specific for factors which act to repress gene transcription that genes may be identified which are exclusively repressed for a variety of transcription factors. Other repressor proteins thought to be recruited by DNA binding transcriptional repressors contemplated by the present invention and which may be utilized as targets for sequential immunoprecipitation include, but are in no way limited to SMRT, SunCoR, FunCoR, SIN1, Sin3A (1), Sin3A (2), Sin3A (3), Sin3B, HP1 and PcG (polycomb group proteins). In addition, proteins which bind selectively to methylated DNA are speculated to be involved in mediating or playing a role in transcriptional repression and/or long-term silencing. Thus these proteins serve as candidates for sequential immunoprecipitation to discover target genes actively repressed by certain transcription factors. The proteins covered by the present invention for the purposes of identifying repressed or silenced transcription factor target genes include, but are in no way limited to the methyl DNA binding proteins MeCP1, MeCP2, MBD1, MBD2, MBD3 and MBD4. Other repressor proteins which have yet to be identified may also ultimately be targeted for sequential immunoprecipitation to define transcriptional repressor target genes.

In addition to the utilization of antibodies specific for the above mentioned and other proteins which may be recruited by specific transcription factors to actively repress genes, antibodies are also contemplated by the present invention which bind specifically proteins that cause modifications in the DNA and or core proteins in chromatin. These modifications include, but are in no way limited to methylation of CpG islands, deacetylation and phosphorylation of histones. Proteins involved in chromatin modification of this sort covered by the presently described invention include, but are in no way limited to HDAC1, HDAC2, HDAC3, HDAC4, HDAC5, HDAC6, HDAC7, HDAC8 and any other as yet undiscovered or uncharacterized proteins which effectively modify chromatin.

As evidenced above, it is the combination of immunoprecipitation with antibodies to tissue or cell type-specific factors, only one example of which is p53, with those specific for the core transcriptional machinery which allows for elimination of nontranscribed sequences and provides for optimal efficient recovery of transcription factor target genes. Antibodies utilized by the present invention specific for the transcription factor p53 described in FIG. 8 are of polyclonal goat origin and recognize the full length protein (cat #sc6243, Santa Cruz Biotechnology, Inc.). It is clear that sequential precipitation of cross-linked chromatin can be performed for a variety of tissue and cell type-specific transcription factors for the purposes of ultimately identifying both known and unknown target loci for these factors. While the presently described invention demonstrates its applicability to the discovery of both known and previously undiscovered target loci for the transcription factor p53, it is in no way limited in its utility for this particular transcription factor. Other transcription factors of prokaryotic, eukaryotic and viral origin contemplated and covered by the present invention include, but are not limited to A2, AAF, abaA abd-A, Abd-B, ABF1, ABF-2, ABI4, Ac, ACE2, ACF, ADA2, ADA3, ADA-NF1, Adf-1, Adf-2a, Adf-2b, ADR1, AEF3, AF-1, AF-2, AFLR, AFP1, AFX-1, AG, AG1, AG2, AG3, AGIE-BP1, AGL11, AGL12, AGL13, AGL14, AGL15-1, AGL15-2, AGL17, AGL2, AGL3, AGL4, AGL6, AGL8, AGL9, AhR, AIC3, AIC2, AIC3, AIC4, AIC5, AID2, AIIN3, ALF1B, ALL-1, alpha-1, alpha2uNF1, alpha2uNF2, alph2uNF3, alpha-CP1, alpha-CP2a, alpha-CP2b, alpha-factor, alphaH0, alphaH2, alphaH3, alpha-IRP, alpha-PAL, alpha2uNF1, alpha2uNF3, alphaA-CRYBP1, alphaH2-alphaH3, alphaMHCBF1, Alx-3, Alx-4, ALY, AMDA, AmdR, aMEF-2, AML1, AML1a, AML1b, AML1c, AML1DeltaN, AML2, AML3, AMT1, AMY-1L, A-Myb, AN2, AnCF,ANF, ANF-2, ANR1, Antp, AP-1, AP-2, AP-2alphaisoform2, AP-2alphaisoform3, AP-2alphaisoform4, AP-3, AP3-1, AP3-2, AP4, AP-5, APC, APETALA1, APETALA3, AR, ARA, AREA, AREB6, ARG RI, ARG RII, armadillo, Arnt, ARP-1, ARP7, ARP9, ARR1, AS-C T3, AS321, ASF-1, ASH-1, ASH-3b, ASP, AT-13P2, ATBF1-A, ATBP, AT-BP1, AT-BP2, ATF, ATF-1, ATF-3, ATF-3deltZIP, ATF-adelta, ATF-like, Athb-1, Athb2, Ato, Axial, AZF1, B factor, B″, BAF1, B-TFIID, band I factor, BAP, Barx-1, BAS, BBF1, BBF2a, BBF3, BBFa, Bcd, BCFI, Bcl-3, BCL-6, BD73, BDF1, beta-1, BETA1, BETA2, beta-catenin, beta-factor, BF-1, BF-2, BGP1, Bin1, Blimp-1, BmFTZ-F1, B-Myb, B-Myc, BP1, BP2, B-Peru, BR-C Z1, BR-C Z2, BR-C Z4, Brachyury, BRF1, Br1A, Brn-3a, Brn4, Brn-5, BUF1, BUF2, BAF1, BAS1, BCFII, beta-factor, BETA3, BLyF, BP2, BR-C Z3, brachyuray, brahma, BRF1, Brn1, Brn2, Brn-3a, Brn-3b, Brn4, Brn-5, Bro, Btd, BTEB, BTEB2, BUF, BUF1, BUF2, BUR6, byr3, BZIP910, BZIP911, c-abl, c-Ets-1, c-Ets-2, c-Fos, c-Jun, c-Maf, c-myb, c-Myc, c-Qin, c-Rel, C/EBP, C/EBPalpha, C/EBPbeta, C/EBPdelta, C/EBPepsilon, C/EBPgamma, C1, CAC-binding protein, CACCC-binding factor, Cactus, Cad, CAD1, CAF17, CAL, CAP, CAR2, CArG box-binding protein, CAT8, CAUP, CBF1, CBF2, CBF3, CBF4, CBF5, CBF-A, CBF-B, CBF-C, CBP, CBTF, CCAAT-binding factor, CCBF, CCF, CCG1, CCK-1a, CCK-1b, CCR4, CD28RC, CDC10, Cdc68, CDF, cdk2, CDP, CDP2, Cdx-1, Cdx-2, Cdx-3, Cdx4, CEBF, CEF1, ceh-1, ceh-10, ceh-12, ceh-13, ceh-14, ceh-16, CEH-18 and (all ceh related factors), CeMyoD, c-Ets-1, C-Ets-1A, c-Ets-1B, CF1, Cf1a, CF2-I, CF2-II, CF2-III, CFF, CG-1, CHA4, CHOP-10, Chox-2.7, Chx10, CIN5, CIIIB1, c-Jun, CKB3, Clox, c-Maf, CMB1, CMB2, c-Myb, c-Myc, CNBP, Cnc, CoMP1, core-binding factor, CoS, COUP, COUP-TF, CP1, CP1A, CP1B, CP1C, CP2, CPBP, CPC1, CPE binding protein CPRF-1, CPRF-2, CPRF-3, CPM10, CPM5, CPM7, CPPI, CPRF-1, CPRF-2, CPRF-3, CPRF4a, CPRF-4b, all CREB related factors, CRE-BP1, CRE-BP2, CRE-BP3, CRE-BPa, CreA, CREB, CREB-2, CREBomega, CREMalpha, CREMbeta, CREMdelta, CREMepsilon, CREMgamma, CREMtaualpha, CRF,all CRM related factors, Croc, Crx, CRZ1, CSBP-1, CtBP, CTCF, CTF, CUM1, CUM10, CUP2, CUP9, CUS1, Cut, Cux, CWH-1, CWH-2, CWH-3, Cx, cyclin A, cyclin T, cyclin T1, cyclin T2, cyclin T2a, cyclin T2b, CYS3, D-MEF2, Da, all DAL related factors, DAP, DAPI, DAT1, DAX1, DB1, DBP-A, DBF4, DBP, DBSF, dCREB, DDB, DDB-1, DDB-2, dDP, dE2F, DEAP3, DEF, DEFH2, Delilah, delta factor, deltaCREB, deltaE1, deltaEF1, deltaMax, DENF, DENF1, DENF2, DENF3, DEP, DEP2, DBP3, DEP4, DERmo-1, DF-1, DP-2, DF-3, Dfd, dFRA, DHR3, DHR38, DHR78, DHR96, dioxin receptor, dJRA, D1, DII, all Dlx related factors, DM-SSRP1, DMLP1, Dof3, DP-1, DP-2, Dpn, Dr1, all DREB related factors, DRF1, DRF2, DRTF, DSC1, DSIF, DSP1, DST1, DSXF, DSXM, DTF, E, E1A, E2, E2BP, E2F, E2F-BF, E2F-I, E4, E47, E4BP4, E4F, E4TP2, E7, E74, E75, EAP1, EAP2L, EAP2S, EAR2, EBF, EBF1, EBNA, EBP, EBP40, EC, EC5, ECF, ECF2, ECF3, ECH, ECM22, EcR, eE-TF, EF-1A, EF-C, EF1, EFgamma, EGM1, EGM2, EGM3, Egr, EGR2, EGR3, eH-TF, EIIa, EivF, EKLF, Elf-1, Elg, Elk-1, ELP, Elt-2, EmBP-1, embryo DNA binding protein, Emc, EMF, EMF2, EMP3, EMP4, Ems, Emx, Erx-1, Emx-2, En, ENH-binding protein, ENKTP-1, epsilonF1, ER, Erbeta, EREBP-1, EREBP-2, EREBP-3, EREBP4, ERF1, Erg, Esc, Esc1, esg, Esx-1a, Esx-1b, FM, ETL, Eve, Evi, Evx, Exd, Ey, en-1, en-2, f(alpha-f(epsilon), P27E5.2, F2F, FACB, F-ACT 1, factor 1, factor 2, factor 3, factor B1, factor B2, factor delta, factor I, FAR, Fbf1, FBF-A1, FBP, FBP1, FBP11, FBP2, FBP6, FBP7, f-EBP, FHL1, FIM, FKBP59, Fkh, FKH1, Fkh-1, FKH2, Fkh-2, Fkh-3, Fkh4, Fkh-5, Fkh-6, FKHR, FKHRL1, FKHRL1P1, FKHRL1P2, FKHRP1, FlbD, FLC, FLF, Flh, Fli-1, FLO, FLO8,FLV-1, FOG, FosB, FosB/SF, Fra-1, Fra-2, Freac-1, Freac-10, Freac-2, Freac-3, Freac4, Freac-5, Freac-6, Freac-7, Freac-8, Freac-9, FRG Y1, FRG Y2, FTF, FTS, Ftz, FTZ-F1, FTZ-F1beta, FZF1G factor, G factor, G/HBF-1, G10BP, G6 factor, GA-BP, GABP, GABP-alpha, GABP-beta1, GABP-beta2, GAF, GAF1, GAF2, GAG2, GAL11, GAL4, GAL80, GammaCAAT, gammaCAC1, gammaCAC2, gamma-factor, gammaOBP, GAMYB, GAT1, GAT2, GAT3, GAT4, GATA-1, GATA-1A, GATA-1B, GATA-2, GATA-3, GATA4, GATA-5, GATA-5A, GATA-, GATA-6, GATA-6A, GATA-6B, GBF, GBF1, GBF12, GBF1A, GBF1B, GBF2, GBF2A, GBF2B, GBF3, GBF4, GBF9, GBP, GC1, GC2, GC3, GCF, GCM, GCMa, GCMb, GCN4, GCN5, GCNF, GCR1, GCR2, GE1, GEBF-I, GF1, GFI, Gfi-1, GFII, GHF3, GHF-5, GHF-7, GIS1, GKLF, GL1, Gl15, G12, Glass, GLI, GLI3, GLN3, GLO, GM-PBP-1, GP, GR, GR alpha, GR beta, GRF-1, Grg-4, Grg-5, GRIP1, Groucho, Gsb, GSBF1, Gsbn, Gsc, Gsc A, Gsc B, Gt, GT-1, GT-2, GT-IC, GT-IIA, GT-IIBalpha, GT-IIBbeta, GTS1, Gtx, GZP3, H16, H1TF1, H1TF2, H2B abp 1, H2RIIBP, H4TF-1, H4TF-2, HAC1, HAL9, HALF-1, HAP1, HAP2, HAP3, HAP4, HAP5, Hb, HB9, HBLF, HBP-1, HBP-1a, HBP-1a(11), HBP-1a(c14), HBP-1b, HBP-1b(c1), HCM1, HDaxx, heat-induced factor, HE, HEB1-p67, HEB1-p94, HEF-1B, HEF-1T, HE-4C, HEN1, HEN2, HeRunt-1, HES-1, HES-2, HES-3, HES-5, Hesx1, Hex, HFH-1, HFH-11A, HFH-11B, HFH-2, HFH-3, HFH4, HFH-5, HFH-6, HFH-7, HFH-8, HIF-1, HIF-1alpha, HIF-1beta, HiNF-A, HiNF-B, HiNF-C, HiNF-D, HiNF-D3, HiNF-E, HiNF-M, HiNF-P, HIP1, HIR1, HIR2, HIR3, HIRA, HIV-EP2, Hlf, Hlf-alpha, Hlf-beta, HLX, Hlx, HMBP, HMG I, HMG I(Y), HMG Y, HMGI-C, HMS1, HMS2, HNF-1, HNF-1A, HNF-1B, HNF-1C, HNF-3, HNF-3(-like), HNF-3alpha, HNF-3B, HNF-3beta, HNF-3gamma, HNF-4, HNF-4(D), HNF-4alpha1, HNF-4alpha2, HNF-4alpha3, HNF-4alpha4, HNF-4alpha7, HNF-4beta, HNF-4gamma, HNF-6, HNF-6alpha, HNF-6beta, hnRNP K, Hox11, HOXA1, HOXA10, HOXA10 PL2, HOXA11, HOXA13, HOXA2, HOXA3, HOXA4, HOXA5, HOXA6, HOXA7, HOXA9, HOXB1, HOXB2, HOXB3, HOXB4, HOXB5, HOXB6, HOXB7, HOXB8,HOXB9, HOXC10, HOXC11, HOXC12, HOXC13, HOXC4, HOXC5, HOXC6, HOXC6 (PRI), HOXC6 (PRII), HOXC8, HOXC9, HOXD1, HOXD10, HOXD11, HOXD12, HOXD13, HOXD3, HOXD4, HOXD8, HOXD9, HP1 site factor, Hp55, Hp65, HrpF, HSE-binding protein, HSF, HSF1, HSF2, HSF24, HSF30, HSF8, hsp56, Hsp90, HST, HSTF, HY5, IBF, IBP-1, IBR, ICER, ICER-I, ICER-Igamma, ICER-II, ICER-Iigamma, ICP4, ICSBP, Id1, Id1.25, Id1H′, Id2, Id3, Id3/Heir-1, Id4, IDS1, IE1, IEBP1, IEFga, IF1, IF2, IFH1, IFNEX, IgPE-1, IgPE2, IgPE-3, Ik-1, Ik-2, Ik-3, Ik4, Ik-5, Ik-6, Ik-7, Ik-8, IkappaB, IkappaB-alpha, IkappaB-beta, IkappaB-gamma, IkappaB-gammal, IkappaB-gamma2, IkappaBR, IK13, ILF, ILRF-A, IME1, IME4, INO2, INO4, INSAF, IPF1, I-POU, IRBP, IRE-ABP, IREBF-1, IRF-1, IRF-2, IRF-3, irlB -2a, Irx-3, ISGP-1, ISGF-3, ISGF-3alpha, ISGF-3gamma, Is1-1, ISRF, ISRFI, ITF, ITF-1, ITF-2, IUF-1, Ixr1, JRF, Jun-D, JunB, JunD, K06B9.5, K07C11.1, kappaY factor, KAR4, KBF2, kBF-A, KBP-1, KCS1, KER1, −1, Kid-I, Kin17, KN1, Kni, Knox3, KNRL, Koxl, Kr, Kreisler, KRP-1, Krox-20, Krox-24, Ku autoantigen, KUP, Lab, LAC9, LBP, LBP-1,

LBP-1a, Lc, LCR-F1, LD, Ldb1, LEF-1, LEF-1B, LEF-1S, LEU3, LF-A1, LF-A2, LF-B2, LF-C, LFY, LG2, LH-2, Lhx-3, Lhx-3a, Lhx-3b, Lhx-4, LHY, Lim-1, Lim-3, lin-1, lin-11, lin-14A, lin-14B1, lin-14B2, lin-29A, lin-29B, lin-31, lin-32, lin-39, LIP15, LIP19, LIT-1,LKLF, Lmo1, Lmo2, Lmx-1, L-Myc1, L-Myc-1, L-Myc-1(ong form), L-Myc-1(short form), L-Myc-2, LR1, LSF, LSIRF-2, LUN, Lva, LVb-binding factor, LVc, LXRalpha, LyF-1, Lyl-1, LYS14, Lz, M factor, M-Twist, M1, m3, Mab-18, MAC1, Mad, MAF, MafB, MafP, MafG, MafK, Mal63, MAPF1, MAPF2, MASH-1, MASH-2, mat-Mc, mat-Pc, MATal, MATalpha1, MATalpha2, MATH-1, MATH-2, Max1, M factor, M1, m3, Mab-18 (284 AA), Mab-18 (296 AA), mab-5, MAC1, Mad1, Mad3, Mad4, MADS1, MADS11, MADS16, MADS2, MADS24, MADS3, MADS4, MADS45, MADS5, MADS6, MADS7, MADS8, MADS9, MAF, MafB, MafF, MafG, MafK, MAL13, MAL23, MAL33, MAL63, MAPF1, MAPF2, MASH-1, MASH-2, Mat1-Mc, MATa1, MATalpha1, MATalpha2, MATH-1, MATH-2, mat-Pc, Max, Max1, Max2, MAZ, MAZi, MB67, MBF1, MBF-1, MBF2, MBF3, MBF-I, MBP1, MBP-1 (1), MBP-1 (2), MBP-2, MCBF, MCM1, MCM1+MATalpha1, MDBP, MDBP-2, MDS3, mec-3, MECA, MED11, MED2, MED4, MED6, MED7, MED8, mediating factor, MEF1, MEF-2, MEF-2B, MEF-2B-1, MEF-2B-2, MEF-2B-3, MEF-2B-4, MEF-2C, MEF-2C (433 AA form), MEF-2C (465 AA form), MEF-2C (473 AA form), MEF-2C/delta32 (441 AA form), MEF-2D, MEF-2D (506 AA form), MEF-2D (514 AA form), MEF-2D00, MEF-2D0B, MEF-2DA-0, MEF-2DA-B, MEF-2DA0, MEF-2DAB, Meis-1, Meis-1-1, Meis-1-2, Meis-1-3, Meis-1-4, Meis-1a, Meis-1b, Meis-2a, Meis-2b, Meis-2c, Meis-2d, Meis-3, Meso1, MET18, MET28, MET31, MET32, MET4, Mf2, MF3, MFH-1, Mfh-1, MGA1, Mhox, MHR1, Mi, MIBP1, MIF-1, MIG1, MIG2, Mix.1, Mix.2, Mix.3, Mix.4, Mixer, MA, Miz-1, MKR2, MLP, MM-1, MNB1a, MNB1b, MNF1, MNR2, MOK-2, MOP3, MOT1, MOT3, MP4, MPBF, MR, MRF4, MRR, Msh, MSN1, MSN2, MSN4, Msx-1, Msx-2, MTB-Zf, MTB1, MTF-1, MTH1, Mtll, mtTF1, M-Twist, muEBP-B, muEBP-C2, MUF1, MUP2, Mxi1, MYB A, MYB.PH1, MYB.PH2, MYB.PH3, MYB1, Myb1, all Myb related proteins, MYB-P1, MYBST1, myc-CF1, myc-PRF, MYC-RP, Myef-2, Myf-3, Myf-4, Myf-5, Myf-6, Myn, MyoD, Myogenin, MZF-1, Nab1, Nau, NBF, NC1, NCB2, NDT80, NELF, NeP1, NER1, Net, NeuroD, NF III-a, NP III-c, NF III-e, NF-1, NF-1/L, NF-1/Red1, NF-1A, NF-1A1, NF-1A1.1, NF-1A2, NF-1a3, NF-1A4, NF-1A5, NF-1B, NF-1B1, NF-1B2, NF-1B3, NP-1B4, NF-1C1, NF-1C2, NF-1C4, NF-1X, NF-1X1, NF-1X2, NF-1X3, NP2d9, NF4FA, NF-4FB, NF-4FC, NF-A, NF-A3, NF-AB, NFalpha1, NEalpha2, NPalpha3, NPalpha4, NF-AT, NFAT-1, NF-AT3, NF-Atc, NF-ATc3, NF-Atp, NF-Atx, NF-BA1, NfbetaA, NF-CLE0a, NF-CLE0b, NF-D, NFdeltaE3A, NFdeltaE3B, NFdeltaE3C, NFdeltaF4A, NFdeltaE4B, NFdeltaE4C, Nfe, NF-E, NF-E1b, NF-E2, NF-E2 p45, NF-E3, NF-E4, NFE-6, NF-EM5, NF-Gma, NF-GMb, NF-H1, NF-H2, NF-H3, NFH3-1, NFH3-2, NFH3-3, NFH3-4, NP-IL-2A, NF-IL-2B, NF-InsE1, NF-InsE2, NF-InsE3, NF-jun, NK-kappaB, NF-kappaB(-like), NF-kappaB1, NF-kappaB1 precursor, NF-kappaB2, NF-kappaB2 (p49), NF-kappaB2 precursor, NF-kappaE1, NF-kappaE2, NF-kappaE3, NF-lambda2, NF-MHCIIA, NF-MHCIIB, NF-muE1, NF-muE2, NF-muE3, NF-muNR, NF-ODC1, NF-S, NP-TNF, NF-U1, NF-W1, NF-W2, NF-X, NF-X1, NF-X2NF-X3, NF-Xc, NF-Y, NP-Y′, NF-YA, NF-YB, NF-YC, NF-Zc, NF-Zz, NGFI-B, NGFI-C, NHP-1, NBP-2NHP3, NHP4, NHR1, NIP, NIRA, NIT2, NIT4, Nkx-2.1, Nkx-2.2, Nkx-2.5, NLS1, NMH7, NMHC5, Nmi, N-Myc, N-Myc1, N-Myc2, nob-1A, nob-1B, N-Oct-2alpha, N-Oct-2beta, N-Oct-3, N-Oct4, N-Oct-5a, N-Oct-5b, NOR1, NOT, NOT1, NOT2, NOT3, NOT5, NP-III, NP-IV, NP-TCII, NP-Va, NPX1, NRD I, Nrf1, NRF-1, Nrf2, NRF-2NRF-2betal, NRF-2gamrnal, NRFA, NRG1, NRG2, NRL, NS-1, NSDD, NTF, NTF1, NUC-1, Nur77, NUT1, NUT2, OBF, OBF-1, OBF3.1, OBF3.2, OBF4, OBF5, OBP, OBP1, OC-2, OCA-B, OCSBF-1, OCSTF, Oct-1, Oct-10, Oct-11, Oct-1A, Oct-1B, Oct-1C, Oct-2, Oct-2.1, Oct-2.3, Oct-2.4, Oct-2.6, Oct-2.7, Oct-2.8, Oct-2B, Oct-2C, Oct4, Oct-4A, Oct4B, Oct-5, Oct-6, Oct-7, Oct-8, Oct-9, Octa-factor, octamer-binding factor oct-B2, oct-B3, Oct-R, Odd, ODR7, OG-12, OG-2, OG-9, OHP1, OHP2, Olf-1, OM1, ONR1, Opaque-2, OPM1, OSBZ8, Otd, Otx1, Otx2, Otx4, Ovo, OZP, P (long form), P (short form), P1, p107, p130, p28 modulator, p300, p38erg, p40x, p45, p49erg, p53as, p55, p55erg, p58, p65delta, p67, PAB1, PacC, PAF1, pag-3, PAGL1, pal-1, Pap1+, par-2, Parxis, PARP, Pax-1, Pax-1/9, Pax-1/9 (AmphiPax-1), Pax-1/9-I, Pax-1/9-II, Pax-1/9-III, Pax-1/9-IV, Pax-1/9-V, Pax-1/9-VI, Pax-2, Pax-2.1 , Pax-2.2, Pax-2/5/8, Pax-2a, Pax-2b, Pax-3, Pax-3A, Pax-3B, Pax4, Pax4a, Pax-4c, Pax-4d, Pax-5, Pax-6, Pax-6 (Pax-QNR), Pax-6/Pd-5a, Pax-6 12.1, Pax-6 12.2, Pax6 4.1, Pax-6 4.2, Pax6 J2, Pax-7, Pax-8, Pax-8a, Pax-8b, Pax-8c, Pax-8d, Pax-8e, Pax-8f, Pax-8g, Pax-9, Pax-A, Pax-B, Pb, PBF, PBP, Pbx-1a, Pbx-1b, Pc, PC2, PC4, PC4 p9, PC5, Pcr1, PCRE1, PCT1, PDM-1, PDM-2, PDR1, PDR3, Pdx-1, PEA1, PEA2, PEA3, PEB1, PEBP2, PEBP2alpha, PEBP2alphaA/Osf2, PEBP2alphaA/til-1, PEBP2alphaA/til-1 (Y), PEBP2alphaA/til-1(U), PEBP2alphaA1, PEBP2alphaA2, PEBP2alphaB1, PEBP2alphaB2, PEBP2beta, PEBP2beta1, PEBP2beta, PEBP2beta3, PEBP5, Pep-1, PERIANT, pes-1apes-1b, PF1, PF3, PGA4, PGD1, pha4, PHAN, PHD1, phiAP3, PHO2, PHO4, PHO80, Phox-2, php-3, PI, PI1, PI2, pie-1, PIHbox9, PIP2, Pit-1, Pit-1a, Pit-1b, Pit-1c, Pitx-3, PLE, PLE/DEPH200, PLE/DEFH49, PLE/DEFH72, PLEYSQUA, PLZF, PNPI2, PO-B, pointedP1, pointedP2, Pontin52, pop-1POP2, POTM1-1, pou[c], Pou2, pox neuro, PP1, PP2, PPAR, PPARalpha, PPARbeta, PPARgamma, PPR1, PPUR, PPYR, PR, PR A, PRb, Prd, PRDI-BF1, PRDI-BFc, PREB, Prop-1, protein a, protein b, protein c, protein d, PRP, PSE1, Psx-1, Psx-2, P-TEFb, PTF, PTP1, PF1-alpha, PTF1-beta, PTFalpha, PTFbeta, PTFdelta, PTFgamma, Ptx-1, Ptx-2, Ptx-2B, Pu box binding factor, Pu box binding factor (BJA-B), PU.1, Pu.1, PUB 1, PuF, PUF-I, Pur factor, Pur-1, PUT3, P-wr, PX, PZF1, qa-1F, QBP, QUT1, R, R1, R2, RAD1, Rad-1, RAD18, RAD2, RAF, RAP1, RAP2.5, RAR, RAR-alpha, RAR-alphal, RAR-alpha2, RAR-beta, RAR-beta1, RAR-beta2, RAR-beta3, RAR-beta4, RAR-gamma, RAR-gamma1, RAR-gamma2, RAV1, RAV2, Rax, Rb, RBP60, RBP-Jkappa, Rc, RC1, RC2, RCS1, REB, REB1, Reb1p, RelA, RelB, repressor of CAR1 expression, REV-ErbAalpha, REX-1, RF1, RF2a, RFX, RFX1, RFX2, RFX3, RFX5, RF-Y, RGM1, RGR1, RGT1, RIC1, RIM1, RIP14, RITA-1, RLM1, RME1, RMS1, Ro, Roaz, ROM1, ROM2, RORalpha1, RORalpha2, RORalpha3, RORbeta, RORgamma, Rox, Rox1, ROX3, RPF1, RPGalpha, RPH1, RREB-1, RRF1, RRF2, RRF3, RRN10, RRN11, RRN3, RRN5, RRN6, RRN7, RRN9, RS2, RSC4, RSRFC4, RSRFC9, RSV-EF-II, RTF1, RTG1, RTG2, RTG3, Runt, RVF, Rx, Rx1, Rx2, Rx3, RXR-alpha, RXR-beta, RXR-beta1, RXR-beta2, RXR-gamma, S8, SAP1, SAP-1a, SAP-1b, SBF, SBF-1, Sc, SCBPalpha, SCBPbeta, SCBPgamma, SCD1/BP, SCM-inducible factor, Scr, S-CREM, S-CREMbeta, Sd, Sdc-1, SDS3, SEP1, SEP-1 (1), SEP-1 (2), SEP3,SEF4, SEM-4, SET1,SET2, SF1, SF-1, SF-2, SF-3, SF-A, SFL1, SGC1, SGF-1, SGF-2, SGF-3, SGF4, Shn, SHP, SHP1, SHP2, SIF, SIG1, SIII, SIIII-p110, SIII-p15, SIII-p18, Sim1, Sim2, Six-1, Six-2, Six-3, Six-3alpha, Six-3beta, Six-4, Six-4A, Six-4B, Six-4C, Six-5, Six-6,Skn-1, SKN7, SKO1, SLM1, SLM2, SLM3, SLM4, SLM5, Slp1,slp2, S-Myc, Sn, SN (sienna), Sna, SNF5, SNF6, SNP1, So, SOX-11, SOX-12, Sox-13, SOX-15, Sox-18, Sox-2, Sox-4, Sox-5, SOX-6, SOX-9, Sox-LZ, Sp1, Sp2, Sp3, Sp4, SPA, spE2F, Sph factor, Spi-B, SpOtx, Sprm-1, SpRunt-1, SQUA, SRB10, SRB11, SRB2, SRB4, SRB5, SRB6, SRB7, SRB8, SRB9, SRD1, SRE BP, SREBP-1, SREBP-1a, SREBP-1b, SREBP-1c, SREBP-2, SREP, SRE-ZBP, SRF, SRY, Sry h-1, Sry-beta, Sry-delta, ssDBP-1, ssDBP-2, SSRP1, Staf, Staf-50, STAT, STAT1, STAT1alpha, STAT1beta, STAT2, STAT3, STAT4, STAT5, STAT5A, STATSB, STAT6, STC, SD1, Ste11, STF,12, STE4,STIF1, STEF2, STKA, STM, STP1, Stra13, StuAp, su(f), Su(H), su(Hw), SUM-1, SUP, SVP, SVP46, SWI/SNF complex, SWI1, SWI2, SWI3, SWI4, SWI5, SWI6, SWP,T-Ag, t-Pou2, T3R, T3R-alpha, T3R-alpha1, T3R-alpha2, T3R-beta, T3R-beta1, T3R-beta2, TAB, T-Ag, TAG1, Tal-1, Tal-1beta, Tal-2, TAR factorTat, Tax, TCF, TCF-1TCF-1A, TCF-1B, TCP-1C, TCF-1D, TCP-1E, -1F, TCF-1G, TCF-2, TCF-2alpha, TCF-3, TCP-3B, TCF-3C, TCF-3D, TCF4, TCF-4(K), TCF4B, TCF4E, TCF-A, TCF-B, TCFbeta1, TDEF, TEA1, TEC1, TEF, TEF 1, TEP-1, TEF2, TEF-2, Tel, TF68, TFE3, TFE3-L, TFE3-S, TFEB, TFEC, TFIIA, TFIIA (13.5 kDa subunit), Tf-LF1, Tf-LF2, TF-Vbeta, TGA, TGA1, TGA1a, TGA2, TGA3, TGA6, TgF1, TGGCA-binding protein, TGT3, Th1, THM1, THM18, THM27, THRA1, TIF1, TIF2, TIN-1, TINY, TIP, tI-POU, TLE1, Tll, Tlx, TM3, TM4, TM5, TM6, TM8, TMP, t-Pou2, TR2, TR2-11, TR2-9, TR3, TR4, Tra-1 (long form), Tra-1 (short form), TRAP, TREB-1, TREB-2, TREB-3, TREF1, TREP2, TRF, TRF (2), Trident, TSAP, TSP3, Tsh,TTF-1, TTF-2, TTG1, Ttk 69K, Ttk 88K, TTP,Ttx, ttx-3, TUBF, Twi, TxREF, TyBF, UAY, UBF, UBF1, UBF2, UBP-1, Ubx, UCRB, UCRF-L, UEF-1, UEP-2, UEP-3, UEF-4, UF1-H3beta, UFA, UFB, UFO, UGA3, UHF-1, UME6, unc-30, unc-37, unc-4, Unc-86, URF, URSF, URTF, USF, USF2, vab-3, vab-7, vaccinia virus DNA-binding protein, Vav, Vax-1, Vax-2, VBP, VDR, v-ErbA, VETF, v-Ets, v-Fos, vHNF-1, vHNP-1A, vHNP-1B, vBNF-1C, VITF, v-Jun, v-Maf, Vmw65, v-Myb, v-Myb/v-Ets, V-Myc, v-Myc, Vp1, Vpr, v-Qin, v-Rel, VSF-1, WC1, WC2, Whn, WT1, WT1I, WZF1, X-box binding protein, X-Twist, X2BP, xam1, X-box binding protein, XBP-1, XBP-2, XBP-3, XF1, XF2, XFD-1, XFD-2, XFD-3, XFG20, XGRAF, Xirol, Xiro2, Xiro3, xMEF-2, XPF-1, XrpFI, XW, XX, yan, YB-1, YB-3, Ybx-3, YEB3, YEBP, Yi, YNG2, YPF1, YY1, ZAP, ZEB, ZEM1, ZEM2/3, Zen-1, Zen-2, Zeste, ZF1, ZF2, ZF5, Zfh-1, Zfh-2, Zfp-35, ZID, ZIP-1A, ZIP-2A, ZIP-2B, ZM1, ZM38, Zmhoxla, Zn-15, ZNF174, ZPT2-1, ZPT2-2, ZPT2-3, ZPT2-4, Zta. In addition, any factors which retain the ability to regulate gene expression, either through activation or repression, and are as of yet previously undiscovered or as uncharacterized are covered by the present invention.

While the procedure of sequential immunoprecipitation of cross-linked protein/DNA complexes for purposes of detecting actively transcribed target genes in the presently described invention involves the sequential precipitation of protein/DNA complexes utilizing antibodies specific to the large subunit (c) of RNA polymerase II first and antibodies specific for the transcription factor of interest second, it is in no way limited to this particular order of immunoprecipitation. It is contemplated by the present invention that the immunoprecipitation procedure may be reversed and thus performed with antibodies specific for the transcription factor of interest first and antibodies specific for the large subunit of RNA polymerase II second, although it is possible that a loss of target loci recovery may result due to initial precipitation of genes not activated by said transcription factor of interest.

It is also contemplated and covered by the presently described invention that sequential rounds of immunoprecipitation may be performed with antibodies specific to cell type and tissue restricted transcription factors for the purposes of identifying target genes for multiple factors. For example, the technology described herein may be utilized to search for loci which are targets for regulation by both p53 and Rb, or by both Pit-1 and GATA2 (El-Diery et al., Cell, 1993, 75: 817-825; Dasen et al., Cell, 1999, 97: 587-598). In addition, it is contemplated by the present invention that coimmunoprecipition utilizing antibodies specific for more than one transcription factor simultaneously may be successfully performed for the purposes of identifying target loci for two or more transcription factors.

5.4 Solid Phase Chromosomal Immunoprecipitation Increases both Yield and Sensitivity

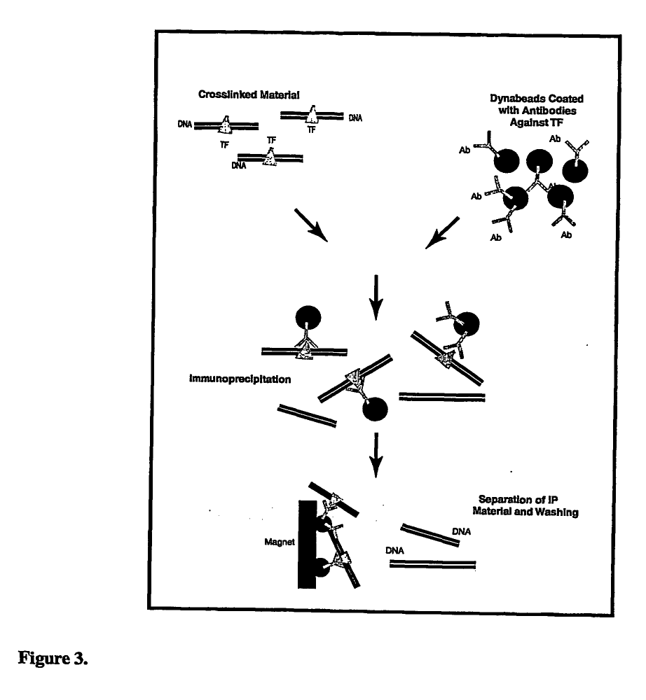

The presently described invention utilizes magnetic beads linked covalently to either monoclonal or polyclonal antibodies specific for discrete and particular transcription factors (Dynal Corporation). It is clear that by implementing solid phase separation techniques for immunoprecipitation both the amount of material recovered as well as the specificity for real in vivo interactions is considerably enhanced. This is due primarily to the increased ability to recover the protein/DNA complexes of limited quantity and implementation of additional washing procedures as compared to immunoprecipitation in the absence of using a solid phase base. A diagrammatic illustration of the use of solid phase technology to increase yield and sensitivity is represented in FIG. 3. Cross-linked DNA/protein material is combined with magnetically charged Dynabeads upon which antibodies to the protein of interest have been conjugated. Use of a magnet results in purification of protein/DNA complexes of interest. Subsequent washing steps allow for the removal of the unbound cellular debris, proteins and DNA fragments. Magnetic bead/protein/DNA complexes are subsequently subjected to further analysis as discussed below. In the presently described invention linkage of antibodies to the solid phase support magnetic beads is accomplished via standard protocol (Dynal Corporation product information and specifications) and those known and skilled in the art are capable of establishing this linkage successfully. Beads are washed briefly in phosphate buffered saline (PBS), pH 7.4. 0.1-1.5 ug of antibody is added per ml of beads, the volume adjusted and the mixture incubated for 24 hours at 4 deg. C. on a rotating shaker. The beads are subsequently collected in test or eppendorph tubes via a magnet and the supernatant removed. After two more rounds of washing in 10 mM Tris-HCl, pH 7.6 for an additional 16-24 hours the bead/antibody complex is ready for sequential immunoprecipitation of protein/DNA complexes.

The particular magnetic beads utilized as a solid phase supporting material in the presently described invention are Dynabeads M-450 Tosylactivated (Dynal Corporation). Other magnetic beads contemplated by the present invention and created by Dynal Corporation which may be utilized as a solid phase support for the chromosomal immunoprecipitation reaction described herein include Dynabeads M-450 uncoated, Dynabeads M-280 Tosylactivated, Dynabeads M-450 Sheep anti-Mouse IgG, Dynabeads M-450 Goat anti-Mouse IgG, Dynabeads M-450 Sheep anti-Rat IgG, Dynabeads M-450 Rat anti-Mouse IgM, Dynabeads M-280 sheep anti-Mouse IgG, Dynabeads M-280 Sheep anti-Rabbit IgG, Dynabeads M-450 sheep anti-Mouse IgG1, Dynabeads M-450 Rat anti-Mouse IgG1, Dynabeads M-450 Rat anti-Mouse IgG2a, Dynabeads M-450 Rat anti-Mouse IgG2b, Dynabeads M-450 Rat anti-Mouse IgG3. Other magnetic beads which are also contemplated by the present invention as providing utility for the purposes of sequential immunoprecipitation include streptavidin coated Dynabeads.

While the presently described invention employs magnetic beads as the solid phase to increase yield and recovery of protein/DNA complexes during sequential chromosomal immunoprecipitation, it is in no way the only solid phase support system which may be implemented successfully to increase yield and sensitivity. Other solid phase supports contemplated by the present invention include, but are not limited to, sepharose, chitin, protein A cross-linked to agarose, protein G cross-linked to agarose, agarose cross-linked to other proteins, ubiquitin cross-linked to agarose, thiophilic resin, protein G cross-linked to agarose, protein L cross-linked to agarose and any support material which allows for an increase in the efficiency of purification of protein/DNA complexes.

An alternative method of attaching antibodies to magnetic beads or other solid phase support material contemplated by the present invention is the procedure of chemical cross-linking. Cross-linking of antibodies to beads may be performed by a variety of methods but may involve the utilization of a chemical reagent which facilitates the attachment of the antibody to the bead followed by several neutralization and washing steps to further prepare the antibody coated beads for sequential immunoprecipitation.

Yet another method of attaching antibodies to magnetic beads contemplated by the pent invention is the procedure of UV cross-linking. A third method of attaching antibodies to magnetic beads contemplated by the present invention is the procedure of enzymatic cross-linking.

The presently described invention implements a solution of solid material in conjunction with antibody/protein/DNA complexes, yet other methodology, such as that which utilizes a column support fixture rather than a solution format may be successfully employed for purposes of solid phase sequential chromosomal immunoprecipitation. In addition, support fixtures such as petry dishes, chemically coated test tubes or eppendorph tubes which may have the capability to bind antibody coated beads or other antibody coated solid phase support materials may also be employed by the present invention.

In the presently described invention the superparamagnetism of the beads allows for the use of a conventional magnet to separate bead/antibody/protein/DNA complexes from nonspecific interactions present with the reaction mixture. The magnetic property of the bead is due to the presence of γFe₂O₃ and Fe₃O₄ found within the bead (Dynal Corporation product information and specifications), although it is also contemplated by the present invention that a number of other materials possessing magnetic properties may be sufficient to confer an ability for efficient separation of bead/antibody/protein/DNA complexes from nonspecific materials in the reaction mixture.

Upon sufficient isolation of protein/DNA complexes utilizing solid phase sequential immunoprecipitation technologies described in the present invention it is necessary to reverse cross-linkages so that DNA fragments containing transcription factor target genes may be precipitated and further manipulated through both standard and modified molecular biology procedures. Those known and skilled in the art are capable of successfully reversing cross-linkages via conventional chromosomal immunoprecipitation protocols. Reversal of cross-linkages is accomplished through an incubation of the isolated protein/DNA complexes at high temperatures, preferably 65 deg. C. for 24 hours. It is also contemplated by the present invention that alternative temperatures may be employed which effectively reverse cross-linkages in the protein/DNA complexes purified. It is anticipated that temperatures higher or lower than 65 deg. C. may also result in a reversal of cross-links. It is speculated that any temperature above 37 deg. C. may, to a certain degree, result in the reversal of chemically driven cross-links. In addition, it is contemplated by the present invention that reversal of cross-linkages through chemical methods such as alkali treatment as well as UV or enzymatic manipulation may be implemented successfully and are covered by the presently described invention for the purposes of solid phase sequential immunoprecipitation and ultimately transcription factor target gene discovery.

Precipitation of DNA fragments containing potential transcription factor target loci is performed in the presently described invention through the use of a typical salt and ethanol mixture (Ausubel et al. (editors), Current Protocols in Molecular Biology, 1994, Chapter 2, p 1-3). Those known and skilled in the art of standard molecular biology procedures are capable of DNA fragment precipitation and collection. It is contemplated that the salt may be omitted without a significant loss in sample recovery. In addition, for the purposes of the presently described invention a coprecipitant is added which allows for visualization of the DNA pellet after precipitation and centrifugation. The coprecipitant Pellet Paint® (Novagen Corporation) has been successfully employed in the present invention for purposes of precipitated DNA visualization and increased recovery (Lunyak et al., Innovations, 2001, 12: 4-5). It is also contemplated by the presently described invention that other coprecipitants may be effectively used to prevent sample loss and increase yield. Polyethylene glycol (PEG) and yeast RNA or any other coprecipitant which effectively acts as a carrier or allows for visualization of the DNA may also be used to accomplish increased yield and minimization of sample loss and are covered by the present invention.

5.5 Modified Inverse PCR in Combination with Sequential Solid Phase Chromosomal Immunoprecipitation Allows for the Discovery of Direct Transcription Factor Targets

Since its inception by Kary Mullis almost 20 years ago, the polymerase chain reaction (PCR) has had a revolutionary impact on the study of DNA in general as well as gene expression in particular (Innis et al., Academic Press, 1990; McPherson et al., IRL Press, 1991; Erlich, A. Stockton Press, 1989; PCR is also illustrated in U.S. Pat. Nos. 4,683,195, 4,683,202, 4,800,159, 4,965,188, 5,023,171, 5,066,584, 5,075,216, 5,091,310, 5,104,792 which are herein incorporated by reference). One significant limitation of PCR is the requirement of the knowledge of nucleotide sequence in order to generate products of unknown sequence internal to those of known origin. The inability to retrieve sequences external to known sequences was overcome by the development of inverse PCR (I-PCR) a method in which circularized DNA provided a template for the successful amplification of sequences of unknown nucleotide composition external to those known (Ochman et al., Genetics, 1988, 120(3): 621-623). Thus it is now possible to retrieve nucleotide sequences adjacent to those of known composition for the purposes of assessing the identity of template DNA.

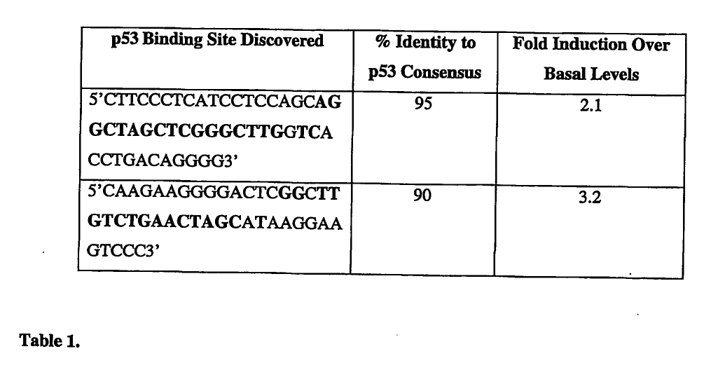

A modified version of the I-PCR technology is described in the present invention which takes advantage of the fact that for many transcription factors the binding site, or at least a consensus binding site, has been characterized through methods such as binding site selection (Ausubel et al. (editors), Current Protocols in Molecular Biology, 1994, Chapter 12, p11/1-11/6). By utilizing degenerate oligonucleotides specific for the binding site of a transcription factor in the format of inverse PCR it is possible to generate flanking sequence information which may aid in determining if the template in question is a target gene for the transcription factor being studied. More specifically, it is now possible to determine if the template is a direct target for transcriptional regulation by the transcription factor being studied. FIG. 9 illustrates the strategy behind the modified I-PCR technology described herein.

While a modified version of I-PCR is described in the present invention, other PCR technologies may be implemented for the rapid retrieval of transcription factor target genes from immunoprecipitated cross-linked protein/DNA complexes as well as DNA purified from these complexes upon reversal of cross-linkages. Other PCR amplification technologies contemplated to be combined with solid phase sequential immunoprecipitation and therefore covered by the present invention include, but are in no way limited to RT-PCR, 5′ RACE (Rapid Amplification of cDNA Ends), 3′ RACE, nested PCR, degenerate oligonucleotide PCR, PCR using oligos coding for transcription factor binding sites in combination with oligos coding for sequences proximal to the transcriptional initiation site such as the TATAA box, and any PCR technology which aids the presently described invention for the purposes of identifying both known and unknown transcription factor target loci.

5.6 Combination of Solid Phase Technology, Sequential Chromosomal Immunoprecipitation, Modified I-PCR and other Molecular Cloning Methods for the Purposes of Transcription Factor Target Gene Discovery

The sensitivity of the methodology relies heavily on the availability of high-quality monoclonal or polyclonal antibodies that can immunoprecipitate the antigen of interest in an efficient and specific manner. The current technology described herein details a method which utilizes antibody coated magnetic beads in combination with the use of a coprecipitant for the precipitation of chosen antigens/DNA complexes with high efficiency and with virtually no background (FIG. 3). Strategies implemented for the further reduction in background nonspecific binding are discussed below.

Originally, the ChIP technology was designed to characterize protein interactions with known DNA target sequences. The methods described herein have been recently developed as to extend the ChIP assay to high-throughput cloning and analysis of both known and unknown sequences, many of which may represent potential transcription factor target loci. FIG. 5 outlines methods developed and optimized for the efficient acquisition of target gene sequence information. DNA cross-linked to binding proteins is isolated from tissue culture cells and sonicated to give the desired fragment length in preparation for immunoprecipitation. Fragments may either be immunoprecipitated (IP'd) directly or “preIP/IP'd” utilizing an antibody to the large subunit of RNA polymerase II that allows for the isolation of only transcribed genes.

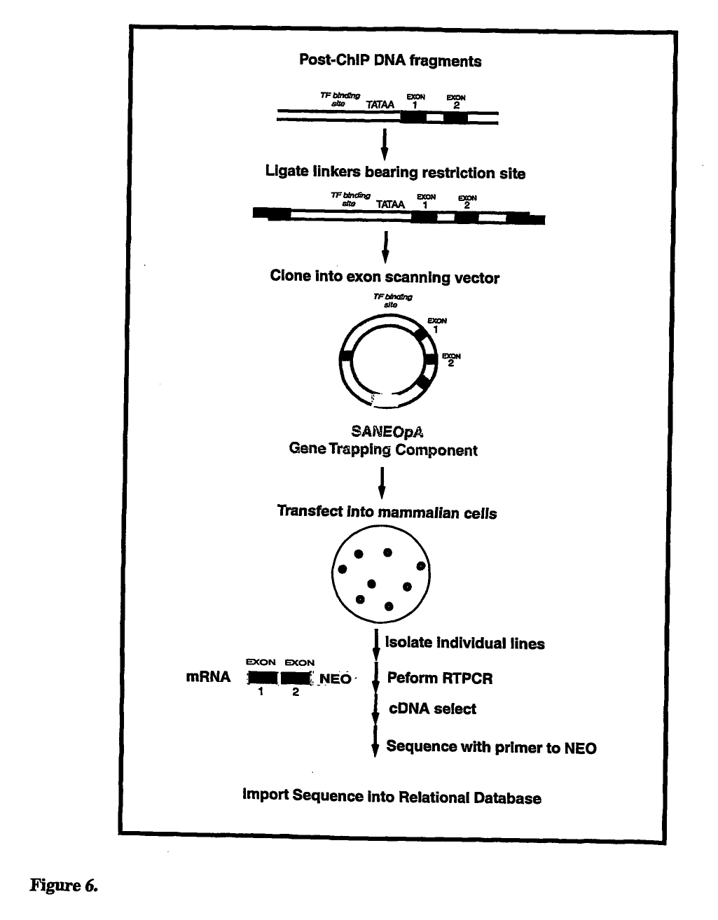

DNA fragments which have been sequentially immunoprecipitated are subsequently run through one or more of a series of sequence acquisition options. Cloning of immunoprecipitated fragments into bacteriophage particles and/or exon scanning vectors allows for high-throughput retrieval of both coding and noncoding individual sequences. FIG. 6 illustrates the process of exon scanning. In addition, the employment of modified inverse PCR methodology described above reveals the possible presence of direct targets for regulation by the transcription factor being studied. By assessing the presence of transcription factor binding sites present within the PCR'd and/or cloned fragments it is possible to draw conclusions as to the possibility of sequences obtained as representing real in vivo targets. This observation is more applicable to factors which have large, invariant and therefore rare binding sites. In addition, it is contemplated and therefore covered by the present invention that computer programs may be used to analyze sequences obtained in search of exons or other revealing attributes such as regulatory elements.

Finally, internal controls such as known genes, binding site information and gel electromobility shift assays (EMSA's) can be used for detailed assessment of signal-to-noise issues (Ausubel et al. (editors), Current Protocols in Molecular Biology, 1996, Chapter 12, p/1-2/5). Through implementation of the technology represented by the presently described invention it is possible to quickly and efficiently implement a saturation screen for target loci for virtually any transcription factor. A demonstration of specific aspects of this technology is evidenced in FIG. 7 for the basal transcription factor RNA Polymerase II. A series of positive internal controls such as immunoprecipitation with antibodies specific for histones as well as background minimization parameters are incorporated into the presently described technology which results in an unsurpassed level of “genome sifting” for genes regulated by transcription factors of either DNA binding or nonDNA binding origin.

Given the fact that background nonspecific chromatin immunoprecipitation could affect the ability to define a considerable percentage of the in vivo targets for the transcription factors being studied, an assessment of background levels is necessary. Experimental procedures have been designed which will evaluate the “signal to noise” ratio for each factor. This is possible by assessing within a defined population of immunoprecipitated chromatin the representation of known genes regulated by the chosen factor. Therefore, for each factor an extensive Southern blot characterization of sample populations of fragments with known target loci as probes may be performed. For example, p53 has previously been shown to be a transcriptional regulator of the p21 locus (El-Diery et al., Cell, 1993, 75: 817-825). The binding site has been thoroughly mapped and sequenced. Thus, it will be possible to utilize this locus as a probe to study in a given population of cloned fragments the percentage which hybridize to this particular probe. Calculation of background can then be performed by assessing the percentage of the population which represents said known target and extrapolation from the predicted number of targets for p53. For example, by assuming a reasonable number of direct targets for p53 at between 30-50 (i.e. for genes involved primarily in regulating proliferation, not apoptosis) it is possible to calculate the efficiency of the system.

5.7 The Creation of a Compendium of Transcription Factor Targets

Application of the improved solid phase sequential chromosomal immunoprecipitation technologies combined with standard and modified molecular biology procedures described herein allows for the collection of DNA fragments putatively containing target loci for any of a multitude of both DNA binding and nonDNA binding transcription factors. Said collection of DNA represents valuable nucleotide sequence information which may reveal novel gene cascades and ultimately therapeutic targets or strategies for the efficient design of therapeutics to treat a variety of human anomalies. The genetic cascades and protein entities encoded by loci representing transcription factor target genes will undoubtedly reveal novel mechanisms of therapeutic intervention.

It is speculated that the collection of nucleotide sequence information obtained through implementation of the presently described invention or technologies described herein may be organized into a searchable database format. This is particularly applicable with respect to each transcription factor or with respect to the discrete realms of human physiology and disease which are represented by the transcription factors for which target genes are discovered. Database configuration of nucleotide sequence information for the purposes of therapeutic target discovery is not a new concept and has proven considerably beneficial to the scientific and medical communities (Celera Discovery System™, Celera Genomics, Inc.; Lifeseq™, Incyte Pharmaceuticals, Inc.; Deltabase™, Deltagen, Inc.) (Venter et al., Science, 2001, 291(5507): 1304-1351). Thus the nucleotide sequences of either coding or noncoding origin (i.e. regulatory elements) discovered through implementation of the technology described herein represent a valuable entity which may be mined for therapeutic utility via efficient computer algorithms. Programming languages contemplated by the present invention which may be utilized to create searchable databases of transcription factor target genes include but are in no way limited to C, C+, C++, Visual C++, Basic, Visual Basic, Java, Visual Java, Perl and any other program which effectively annotates sequence information discovered by implementation of the technology described herein. In addition, it is contemplated and therefore covered by the present invention that computer programs may be utilized to search or scan sequences obtained by technology described in the present invention for the purposes of discovery valuable coding sequence or regulatory information. Use of programs such as, but not limited to BLAST, BLASTX, BLASTP, TBLASTN for such purposes are therefore contemplated and covered by the present invention (Altschul et al., J. Mol. Biol., 215: 555-565; National Center for Biotechnology Information, Basic Local Alignment Search Tool computer algorithms and related variations; Ausubel et al. (editors), Current Protocols in Molecular Biology, 1995, Chapter 19, p3/1-3/27).

Below are described several examples which illustrate application of the presently described invention for the purposes of discovering transcription factor target loci. It should in no way be inferred that the below examples represent the only application of the described technology and hence the invention is not to be constrained by these particular examples.

### 6.0 EXAMPLES

6.1 Demonstration of Improved Chromosomal Immunoprecipitation

For some transcription factor target genes DNA binding sites of either a direct or indirect nature may be located very proximal to the basal transcriptional machinery and transcriptional initiation site of target loci. Other sites may be a distance of several kilobases from the promoter region and transcriptional initiation site. Therefore the need for generating DNA fragment lengths of different sizes represents a crucial aspect of the described technology. By varying fragment length it is possible to immunoprecipitate not only DNA molecules containing sites proximal but also distal to the transcriptional initiation region. FIG. 4 illustrates the ability to “customize” DNA fragment length by varying sonication conditions. DNA isolated from cells was sonicated under increasing temporal conditions, run on a 1.2% agarose gel in 0.5× TBE along with molecular weight markers and stained with ethidium bromide. As the length of time for sonication is increased, it is evident that the fragment sizes of crosslinked DNA become smaller. It is this customizable aspect of the described technology which makes is possible to isolate and characterize virtually any transcription factor target gene.

A basic demonstration of the application of the technology described herein focuses on the gene II/9-1 of Sciara coprophila (Gabrusewycz-Garcia, N. and Kleinfeld, R. G., Journal of Cell Biology, 1966, 29(2): 347-359). FIG. 7 reveals the ability to immunoprecipitate target genes utilizing antibodies specific for the large subunit of RNA polymerase II. In vivo CHIP assay reveals an engaged RNA Pol II at the Sciara coprophila gene 11(9-1 promoter during amplification stage of larval development. 25 salivary glands from either preamplification or amplification stages of larval development were dissected from larvae and incubated in Canon's medium containing 1.0% formaldehyde for 15 min at room temperature and for 30 min at 4 degrees C. In vivo fixed isolated chromosomal DNA's were recovered from tissues and digested with Hind III enzyme (Hind III restriction map of DNA Puff II/9A locus is given on the panel (A). Released chromatin fragments were immunoprecipitated with antibodies raised against Drosophila melanogaster second large subunit of RNA Pol II (gAPD-1) (Weeks et al., Genes Dev., 7: 2329-2344) or with monoclonal anti-histone antibodies (Chemicon, Inc.) coated to Dynabeads (Dynal Corporation). The specificity of these antibodies against Sciara coprophila protein extracts was analyzed by Western blot and shown on panel (A) Pellets were washed extensively and freed from cross-links by incubation of pelleted cross-linked protein/DNA complexes at 65 degrees C. overnight. Purified DNA fragments were subjected to 30 cycles of PCR using a primer sets C and B and analyzed on a 2.5% agarose gel. Primer set B was chosen as a control to demonstrate the specificity of immunoprecipitation with antibodies which recognize the large subunit of RNA polymerase II. Given the fact that binding sites for set B are located 2.5 kb upstream of the geneII/9-1 promoter and sonication resulted in 300-500 bp fragments no ORI sequences should be detected. (B) PCR analysis of input DNA's before immunoprecipitation. Equivalent volumes of purified, in vivo formaldehyde-fixed, Hind III digested DNA samples from either preamplification stage (1) or amplification stage (2) without immunoprecipitation were freed of cross-links and analyzed by 30 cycles of PCR by primer set C. (C) ChIP of cross-linked DNA reveals an engaged RNA Pol II at the promoter of gene II/9-1 only during DNA amplification stage (8) but not at the preamplification stage of larval development (7). At the same time formaldehyde cross-linked histones are detectable on a promoter containing DNA fragment during both preamplification and amplification stages of larval development (C). Equal amounts of cross-linked, Hind III-digested DNA material were precipitated either with anti-histone antibodies (lanes 1,2,3,4), anti-Pol II antibodies (lanes 7, 8) or subjected to non-immune precipitation by magnetic beads as a control to monitor nonspecific precipitation of cross-linked complexes (5,6). Samples in lanes 1,2,4,5,6,7,8 were freed of cross-links and 30 cycles of PCR with primer set C were done for each sample. The absence of PCR product in lane 3 demonstrates the necessity of thermal reversal of the cross-links prior to PCR. Lanes 5 and 6 show that no PCR products are detected in non-immune precipitants. (D) Similar ChIP experiments with PCR analysis of a distinct genomic region after IP's were done in order to demonstrate completion of Hind III restriction digestion. No products were obtained by PCR amplification with primer set B in the samples of anti-RNA Pol II IP for both stages. The level of anti-histone pull down is the same (3, 4) as shown by primer set C.

Multiple rounds of immunoprecipitation may result in the reduction of acquisition of significant amounts of DNA template due to loss upon repeated immunoprecipitation and washing. Thus, it is necessary to test the recoverability of target genes both and after sequential immunoprecipitation. FIG. 8 demonstrates both the efficiency and stringency of multiple immunoprecipitation rounds by assessing the quantitative presence of the p21 target gene for the transcription factor p53 both before and after sequential IP at very stages of the process (El-Deiry et al., Cell, 1993, 75: 817-825). Hela cells were grown to 60% confluency on a 100 mm petry dish, irradiated at 0.5 Gry to stimulate a p53 dependent response and incubated for 6 hours at 37 deg. C. and 7.2% CO₂. Cells were cross-linked in 10% Fetal Bovine Serum Medium containing 1.0% formaldehyde for 30 minutes at 4 deg. C. Cells were harvested, lysed and chromatin fragment length was customized to a length of 50-300 bp through implementation of microtip sonication via 9×15 second pulses of a Branson model 250 sonifier with a 5.0 minute incubation on ice between each 15 second pulse. Samples of PCR template were taken at various points during the solid phase sequential immunoprecipitation procedure to assess the presence or absence of the p21 target gene. p21 sequences were detected only in the sonicated sample prior to immunoprecipitation (sample #1) and in the fraction containing cross-linked protein/DNA adducts precipitated by both antibodies recognizing the large subunit of RNA polymerase II and holo p53 (sample #5). Semi-quantitative PCR demonstrates that very little, if any, template is lost after double IP and the implementation of extensive washing conditions. In addition, no detection of the p21 gene was observed in the supernatant after PolII large subunit immunoprecipitation (sample #2), the residual wash of PolII large subunit antibody conjugated beads after immunoprecipitation (sample #3) or the pelleted beads after pH adjustment and reversal of antibody/ligand binding (sample #4).

6.2 Demonstration of Novel p53 Target Gene Capture

In order to demonstrate the utility of the presently described invention transcription factor target sequences were sought for the mammalian tumor suppressor p53 as mentioned above. Specifically, Hela cells exhibiting 60% subconfluency on a 100 mm petry dish were subjected to 0.5 Gry and incubated for 6 hours at 37 deg. C., 7.2% CO₂. Irradiation of cells activates the p53 response to DNA damage and allows for a characterization of target gene activity. Cells were subsequently cross-linked in 1.0% formaldehyde for 20 minutes, neutralized in 100 mM glycine and harvested for lysis. After three rinses in PBS partial lysis was carried out in 200 ul 100 mM Hepes, pH 7.6, 2 mM EGTA, 2 mM EDTA, 2.0% Triton X-100 via 25 strokes through a 25 G needle. After brief centrifugation and removal of supernatant lysis was continued by performing 25 strokes through a 25 G needle in the presence of 200 ul 100 mM Hepes, pH 7.6, 20 mM MgCl2, 3.0% sarcosyl, 150 mM NaCl. After brief centrifugation and removal of supernatant lysis was completed by performing 25 strokes through a 25 G needle in the presence of 200 ul 100 mM Hepes, pH 7.6, 20 mM MgCl, 150 mM NaCl. Upon removal of supernatant samples were dissolved in 100 ul 10 mM Tris, pH 7.6, 5 mM EDTA.

Chromatin fragment length was customized to a length of 50-300 bp through implementation of microtip sonication via 9×15 second pulses of a Branson model 250 sonifier with a 5.0 minute incubation on ice between each 15 second pulse. FIG. 4 illustrates the fragment sizes obtained on a 1.2% agarose gel stained with Ethidium Bromide and analyzed under UV fluorescence.

Solid phase sequential chromosomal immunoprecipitation was performed with superparamagnetic tosylactivated Dynabeads (Dynal Corporation) linked to antibodies specific for the large subunit of RNA polymerase II and holo p53. Antibodies utilized in the current experiment were p53 (FL-393) (cat. #6243, Santa Cruz Biotechnology, Inc.). Fragments containing transcribed sequences were first isolated from sonicated chromatin samples through the use of beads coated with an antibody to the large subunit of RNA Polymerase II (cat. #sc-9001, Santa Cruz Biotechnology, Inc.). 2 ul of antibody coated beads were incubated with 5 ul sonicated sample DNA in 10 ul PBS buffer overnight at 4.0 deg. C. It was at this step that the pH was altered to a value of 5.5 to allow for the release of cross-linked protein/DNA adducts from the antibody-coated beads. Subsequently immunoprecipitation using beads coated with antibodies to p53 was performed as described above. After second round chromosomal immunoprecipitation bead/antibody/protein/DNA complexes were washed 3 times in 500 ul 10 mM Hepes, pH 7.6, 2 mM EGTA, 2 mM EDTA, 2% Triton X-100, 3.0% Empegen (cat. #324690, Calbiochem Corporation). A similar wash was repeated 3 times in the same buffer containing 1.0% Empegen. A final wash was subsequently performed in a similar buffer without Empegen. Proteinase K treatment was performed on samples for 1.0 hour at 50.0 deg. C. by standard protocol. DNA was precipitated via the addition of 250 ul 100% ethanol, 10 ul 2.5 M NaOAc and 2 ul Pellet Paint® coprecipitant (cat. #70748-3, Novagen Corp.).

FIG. 9 illustrates the concept of modified inverse PCR (IPCR) for the purposes of defining transcription factor target loci in the context of sequential chromosomal immunoprecipitation. PCR is possible through the addition of linkers bearing the restriction site and subsequent episomal circularization. The success of the application of I-PCR itself suggests that the DNA fragments isolated may inherently be direct target genes of the transcription factor being studied, in this case p53. In the present example, degenerate oligonucleotide sequences corresponding to the p53 consensus DNA binding site (RRRCWWGYYYRRRCWWGYYY) linked to an EcoR1 restriction site were utilized to perform PCR and obtain flanking nucleotide sequence information (El-Diery et al., Nat Genet., 1992, 1(1): 45-49). 1.0 ug of sequentially immunoprecipitated DNA was blunt ended in the presence of Klenow fragment (cat. #M-0212S, New England Biolabs) and 25 uM dNTP's for 1.0 hour at 37.0 deg. C. After NucTrap™ (cat. #400702, Stratagene Corporation) spin column purification EcoR1 linkers were ligated to the fragments at 4.0 deg. C. overnight. Fragments flanked by restriction sites were kinased in the presence of T4 polynucleotide kinase (cat. #M0201, New England Biolabs) and 10 mM ATP and religated for circularization. 50 ng of circularized template DNA in combination with 100 ng each oligonucleotide was subjected to 25 rounds of PCR under the following conditions: 98.0 deg. C. for 30 seconds, 55.0 deg. C. for 30 seconds and 72.0 deg. C. for 30 seconds. PCR fragments were excised from a 1.2% agarose gel, blunted and shotgun subcloned into the Sma1 restriction site of pBluescript SK. Plasmids containing fragments were sequenced via Sanger dideoxy sequencing methodology. The presence of the EcoR1 linker sequence reveals the outermost flanks amplified by the PCR reaction.

Table 1 reveals two examples of nucleotide sequences obtained by procedures described herein. Each sequence exhibits high sequence identity to the consensus binding site for p53 (bold letters denote nucleotides fitting the p53 binding site consensus). Sequence A reveals similarity to nucleotide sequences present on Homo sapiens chromosome 17, GenBank accession #AC005562. Sequence B reveals homology to sequences present in Homo sapiens BAC clone RPII-557N21, GenBank accession #AC009242. Both genomic sequences obtained by I-PCR were subcloned upstream of a basal promoter linked to the luciferase reporter gene and cotransfected (20 ug each) with a eukaryotic expression vector containing a cDNA coding for human holop53 into Hela cells at 60% confluency. Cells were subsequently harvested 24 hours after transfection for analysis of reporter gene induction. Induction of transcription of the luciferase reporter was observed for both sequences as compared to basal levels (see Table 1) thus confirming the identification of novel enhancer elements regulatable by the transcription factor p53. The proximity of these regulatory elements with respect to transcribed sequences remains to be determined.

### 7.0 REFERENCES

### U.S. PATENT DOCUMENTS

Mullis et al., U.S. Pat. No. 4,683,195, Issued July, 1987
Mullis, U.S. Pat. No. 4,683,202, Issued July, 1987
Mullis et al., U.S. Pat. No. 4,800,159, Issued January, 1989
Mullis et al., U.S. Pat. No. 4,965,188, Issued October, 1990
Ho et al., U.S. Pat. No. 5,023,171, Issued June, 1991
Gyllensten et al., U.S. Pat. No. 5,066,584, Issued November, 1991
Innis et al., U.S. Pat. No. 5,075,216, Issued December 1991
Innis et al., U.S. Pat. No. 5,091,310, Issued February, 1992
Silver et al., U.S. Pat. No. 5,104,792, Issued February, 1992
Peterson et al., U.S. Pat. No. 5,563,036, Issued October, 1996
Habener et al., U.S. Pat. No. 5,858,973, Issued January, 1999
La Thangue et al., U.S. Pat. No. 5,863,757, Issued January, 1999
Waeber et al., U.S. Pat. No. 5,880,261, Issued March, 1999
Kushner et al., U.S. Pat. No. 6,117,638, Issued September, 2000
Burgess et al., U.S. Pat. No. 6,139,833, Issued October, 2000

### OTHER REFERENCES

Achanzar, W. E., Achanzar, K. B., Lewis, J. G., Webber, M. M., Waalkes, M. P. Cadmium induces c-myc, p53, and c-jun expression in normal human prostate epithelial cells as a prelude to apoptosis. Toxicol. Appl. Pharmacol., 2000,64: 291-330.
Altschul, S. F., Gish, W., Miller, W., Myers, E. W, Lipman, D. J., Basic local alignment search tool. J. Mol. Biol., 215: 555-565.
Ausubel, F. M. et al. (editors), Current Protocols in Molecular Biology, John Wiley & Sons and Green PublishingAssociates, Inc., New York, 1989, 1994, 1995, 1996.
Benashski, S. E., King, S. M., Investigation of Protein-Protein Interactions within Flagellar Dynein Using Homobifunctional and Zero-Length Cross-linking Reagents. Methods, 2000, 22: 365-371.
Dasen, J. S., O'Connell, S. M., Flynn, S. E., Treier, M., Gleiberman, A. S., Szeto, D. P., Hooshmand, F., Aggarwal, A. K, Rosenfeld, M. G., Reciprocal interactions of Pit1 and GATA2 mediate signaling gradient-induced determination of pituitary cell types. Cell, 1999, 97: 587-598.
de Belle I, Mercola D, Adamson E D. Cell-based, high-content screen for receptor internalization, recycling and intracellular trafficking. Biotechniques, July 2000;29(1):170-5.
Deligdisch, L., Effects of hormone therapy on the endometrium. Mod. Pathol., 1993, 6(1): 94-106.
Dynal Technical Handbook, Biomagnetic Applications in Cellular Immunology, 1998.
El-Deiry, W. S., Kern, S. E., Pietenpol, J. A., Kinzler, K. W., Vogelstein, B., Definition of a consensus binding site for p53. Nat. Genet., 1992, 1(1): 45-49.
El-Deiry, W. S., Tokino, T., Valculescu, V. E., Levy, D. B., Parson, R., Trent, J. M., Lin, D., Merser, W. E., Kinzler, K. W., Vogelstein, B. WAF1, a potential mediator of p53 tumor supression, Cell, 1993,75: 817-825.
Erlich, A. PCR Technology: Principals and Applications of DNA Amplification, Stockton Press (1989).
Friend, S. H., Horowitz, J. M., Gerber, M. R., Wang, X. F., Bogenmann, E., Li, F. P., Weinberg, R. A., Deletions of a DNA sequence in retinoblastomas and mesenchymal tumors: Organization of the sequence and its encoded protein. Proc. Natl. Acad. Sci. USA, 1987, 84: 9059-9063.
Frohman, 1994, PCR Methods and Applications, 4:S40-S58.
Gabrusewycz-Garcia, N. and Kleinfeld, R. G., A Study of the Nucleolar Material in Sciara Coprophila. Journal of Cell Biology, 1966, 29(2): 347-359
Hecht, A., Strahl-Bolsinger, S., Grunstain, M., Mapping of DNA interaction sites of chromosomal proteins., Crosslinking studies in yeast. Methods Mol. Biol., 1999, 119: 469-479.
Heinzel, T., Lavinsky, R. M., Mullen, T. M., Soderstrom, M., Laherty, C. D., Torchia, J., Yang, W. M., Brard, G., Ngo, S. D., Davie, J. R., Seto, E., Eisenman, R. N., Rose, D. W., Glass, C. K., Rosenfeld, M. G., A complex containing N-CoR, mSin3 and histone deacetylase mediates transcriptional repression. Nature, 1997, 387: 43-48.
Herzig, T. C., Jobe, S. M., Aoki, H., Molkentin, J. D., Cowley, A. W. Jr., Izumo, S., Markham, B. E., Angiotensin II type1a receptor gene expression in the heart: AP-1 and GATA-4 participate in the response to pressure overload., Proc. Natl. Acad. Sci. USA, 1997, 94: 7543-7348.
Hoffmann, A., Oelgeschlager, T., Roeder, R. G., Considerations of transcriptional control mechanisms: Do TIID-core promoter complexes recapitulate nucleosome-like functions? Proc. Natl. Acad. Sci. USA, 1997, 94: 8928-8935.
Innis et al., PCR Protocols: A Guide to Methods and Applications, Academic Press (1990).
Jepsen, K., Hermanson, O., Onami, T. M., Gleiberman, A. S., Lunyak, V., McEvilly, R. J., Kurokawa, R., Kumar, V., Liu, F., Seto, E., Hedrick, S. M., Mandel, G., Glass, C. K., Rose, D. W., Rosenfeld, M. G. Combinatorial roles of the nuclear receptor corepressor in transcription and development. Cell, 2000, 102(6):753-63.
Kazelenellenbogen, B. S., Montano, M. M., Ekena, K., Herman, M. E., McInerney, E. M. William L. McGuire Memorial Lecture. Antiestrogenes: mechanisms of action and resistance in breast cancer. Breast Cancer Res. Treat., 1997, 44: 23-38.
Kuras, L., Kosa, P., Mencia, M., Struhl, K., TAF-containing and TAF-independent forms of transcriptionally active TBP in vivo., Science, 2000, 19: 1244-1248.
Levine, A. J., Momand, J., Finlay, C. A. The p53 tumor suppressor gene. Nature, 1991, 351: 453-456.
Lockyer, A. E., Jones, C. S., Noble, I. R., Rollinson, D., Use of differential display to detect changes in gene expression in the intermediate snail host Biomphalaria glabrata upon infection wuth Schistosoma mansoni., Parasitology, 2000, 120: 399-407.
Lunyak, V., Baidya, R., Burgess, R., Enhanced sensitivity of ChIP through the application of Pellet Paint® Co-Precipitant, Innovations, 2001, 12, 45.
Maehara, Y., Oki, E., Abe, T., Tokunaga, E., Shibahara, K., Kakeji, Y., Sugimachi, K., Overexpression of the heat shock protein HSP70 family and p53 protein and prognosis for patients with gastric cancer, Oncology, 2000, 58: 144-151.
McKinsey, T A, Olson, E. N., Cardiac hypertrophy: sorting out the circuitry. Curr. Opin. Genet. Dev., 1999., 9: 267-274.
McPherson, M. J. et al., PCR: A Practical Approach, IRL Press (1991).
Molhar, A., Georgopoulos, K., The Ikaros gene encodes a family of functionally deverse zink finger DNA-binding proteins. Mol. Cell Biol., 1999, 14: 8292-8303.
Neilson, L., Andalibi, A., Kang, D., Coutifaris, C., Strauss, J. F., Stanton, J. A., Green, D. P., Molecular phenotype of the human oocyte by PRC-SAGE. Genomics, 2000, 63: 13-24.
Nichogiannopoulou, A., Trevisian, M., Friedrich, C., Georgopoulos, K., Ikaros in hemopoietic lineage determination and homeostasis. Semin. Immunol., 1998, 10: 119-125.
Nikolov, D. B., Burley, S. K., RNA polymerase II transcription initiation: A structural view. Proc. Natl. Acad. Sci. USA, 1997, 94: 15-22.
Ochman, H, Gerber A S, Hartl, D. L., Genetic applications of an inverse polymerase chain reaction. Genetics, November 1988;120(3): 621-3.
Oliner, J. D., Pietenpol, J. A., Thiagalingam, S., Gyuris, J., Kinzler, K. W., Vogelstein, B., Oncoprotein MDM2 conceals the activation domain of tumor supressor p53. Nature, 1993, 362: 857-860.
Rhodes, S. J., DiMattia, G. E., Rosenfeld, M. G., Transcriptional mechanisms in anterior pituitary cell differentiation., Curr. Opin. Genet. Dev., 1994, 4: 709-717.
Sambrook et al. (1989) Molecular Cloning Vols. I-III, Cold Spring Harbor Laboratory Press, Cold Spring Harbor, N.Y.
Scully, K. M., Jacobson, E. M., Jepsen, K., Lunyak, V., Viadiu, H., Carriere, C., Rose, D. W., Hooshmand, F., Aggarwal, A. K., Rosenfeld, M. G. Allosteric effects of Pit-1 DNA sites on long-term repression in cell type specification. Science, 2000, 290(5494): 1127-31.
Semenza, G. L., Transcriptional Regulation of Gene Expression: Mechanisms and Pathophysiology, Human Mut., 1994, 3: 180-199.
Seto, E., Usheva, A., Zambetti, G. P., Momand, J., Horikoshi, N., Weinmann, R., Levine, A. J., Shenk, T. Wild-type p53 binds to the TATA—binding proteins and repress transcription. Proc. Natl. Acad. Sci. USA, 1992, 89: 12028-12032.
Smith, M. L., Kontny, H. U., Bortnick, R., Fornace, A. J. Jr.,The p53-regulated cyclin G gene promotes cell growth: p53 downstream effectors cyclin G and Gadd45 exert different effects on cisplatin chemosensitivity, Exp. Cell Res., 1997, 230: 61-68.
Solomon, J., Larsen, P. L., Varshavsky, A., Mapping Protein-DNA interactions in vivo with formaldehyde: evidence that histone H4 is retained on a highly transcribed gene, Cell, 1988, 53: 937-947.
Stephan, D. A., Chen, Y., Jiang, Y., Malechek, I., Gu, J. Z., Robbins, C. M., Bithner, M. L., Morris, J. A., Carstea, B., Meltzer, P. S., Adler, K., Garlick, B., Trent, J M., Ashlock, M. A., Positional cloning utilizing Genomic DNA microarrays: The Neimann-Pick Type C as a model system. Mol. Gen. Metab., 2000, 70: 10-18.
Tenbaum, S., Baniahmad, A. Nuclear receptors: structure, function and involvement in disease. Int. J. Biochem. Cell Biol., 1997, 29: 1325-1341.
Tjian, R., Maniatis, T. Transcriptional activation: a complex puzzle with few easy pieces. Cell, 1994, 77: 5-8.
Treier, M., Gleiberman, A. S., O'Connell, S. M., Szeto, D. P., McMahon, J. A., McMahon, A. P., Rosenfeld, M. G., Multistep signaling requirements for pituitary organogenesis in vivo. Genes Dev., 1998, 12: 1691-1704.
Venter et al., The sequence of the human genome. Science, 2001, 291(5507): 1304-1351.
Weeks, J. R., Hardin, S. E., Shen, J., Lee, J. M., Greenleaf, A. L., Locus-specific variation in phosphorylation state of RNA polymerase II in vivo: correlations with gene activity and transcript processing, Genes Dev., 1993, 7: 2329-2344.
Winandy, S., Wu, P., Georgopoulos, K., A dominant mutation in the Ikaros gene leads to rapid development of leukemia and lymphoma, Cell, 1995, 83: 289-299.
Wu, W., Cogan, J. D., Pfaffle, R. W., Dasen, J. S., Frisch, H., O'Connell, S. M., Flynn, S. E., Brown, M. R., Mullis, P. E., Parks, J. S., Phillips, J. A. 3rd, Rosenfeld M G. Mutations in PROP1 cause familial combined pituitary hormone deficiency, Nat. Genet., 1998,18: 147-9.
Zambetti, G. P., Bargonetti, J., Walker, K., Prives, C., Levine, A. J. Wild-type p53 mediates positive regulation of the gene expression through a specific DNA sequence element., 1992, Genes Dev., 6: 1143-1152
Zauberman, A., Lupo, A., Oren, M., Identification of p53 target genes through inmmune selection of genomic DNA: the cyclin G gene contains two distinct p53 binding sites, Oncogene, Jun. 15, 1995;10(12): 2361-6.

What is claimed is:

1. A method which utilizes chromosomal immunoprecipitation procedures for the discovery and characterization of transcription factor target genes.

2. A method according to claim 1 comprising a process of:
a) attaching a protein binding entity to a support matrix;
b) utilizing the support matrix/protein binding entity described in a) for the purposes of purifying protein/DNA complexes such as chromatin from cell extracts.

3. A method according to claim 2 wherein said protein binding entity is an antibody.

4. A method according to claim 1 in which said transcription factors are of a DNA binding nature.

5. A method according to claim 1 in which said transcription factors are recruited to DNA through contact with other proteins.

6. A method according to claim 1 in which said transcription factor target genes consist of coding sequences.

7. A method according to claim 1 in which said transcription factor target genes consist of noncoding sequences, including regulatory elements.

8. A method according to claim 7 in which said noncoding sequences are regulated by transcription factors.

9. A method according to claim 2 comprising a process of multiple sequential rounds of immunoprecipitation utilizing protein binding entities of differing origin which involves the process of:
a) immunoprecipitation of cross-linked protein/DNA complexes utilizing protein binding entities specific for one protein followed by;
b) a second round of immunoprecipitation of protein/DNA complexes utilizing complexes isolated by the first round as the substrate and protein binding entities specific for a different protein.

10. A method according to claim 9 which comprises more than two rounds of chromosomal immunoprecipitation.

11. A method according to claim 9 wherein said protein binding entity is specific for members of the basal transcriptional machinery.

12. A method which includes utilizing purified DNA fragments isolated by chromosomal immunoprecipitation procedures described in claim 1 to cross hybridize against libraries of nucleotide sequences.

13. A method which includes utilizing purified DNA fragments isolated by chromosomal immunoprecipitation procedures described in claim 1 to screen against arrays of nucleotide sequences.

14. A method according to claim 1 which includes the implementation of inverse PCR for the discovery of sequences directly bound by said transcription factors.

15. A method according to claim 1 which includes cloning of purified DNA fragments isolated by chromosomal immunoprecipitation into vectors for purposes of manipulation and sequence determination.

16. A protein/DNA complex isolated from cells according to methods described in claim 1.

17. DNA fragments isolated from protein/DNA complexes described in claim 16.

18. Nucleotide sequences present in DNA fragments described in claim 17 wherein said sequences represent noncoding sequences which may include 5 prime and 3 prime untranslated regions, introns, promoter, enhancer and/or silencer elements.

19. Nucleotide sequences present in DNA fragments described in claim 17 wherein said sequences represent coding sequences which correspond to specific amino acid sequences present in putative proteins.

20. Amino acid sequences encoded by nucleotide sequences described in claim 19.

21. Proteins represented by amino acid sequences described in claim 20.

22. A database of sequence information formed from isolated sequences by methods according to claim 1 which represents a cohesive organization of transcription factor target genes.
